(19) United States
(12) Patent Application Publication
(10) Pub. No.: US 2003/0129751 A1
(43) Pub. Date: Jul. 10, 2003

(54) Tissue-engineered organs

(76) Inventors: Tracy C. Grikscheit, Boston, MA (US); Jennifer Ogilvie, Boston, MA (US); Joseph P. Vacanti, Boston, MA (US)

Correspondence Address: FROMMER LAWRENCE & HAUG, 745 FIFTH AVENUE- 10TH FL., NEW YORK, NY, 10151, US

(21) Appl. No.: 10/150,828

(22) Filed: May 16, 2002

Related U.S. Application Data
(60) Provisional application No. 60/343,676, filed on Dec. 27, 2001.
(60) Provisional application No. 60/291,273, filed on May 16, 2001.

(51) Int. Cl. A61F 2/04

(52) U.S. Cl. 435/378; 435/396; 623/23.71; 623/915

(57) ABSTRACT

The present invention relates to a method for producing a tissue-engineered organ or organ portion or specific section thereof comprising the steps of loading organoid units into a biocompatible polymer scaffold and implanting the polymer scaffold into a subject. Organs produced by this method are also encompassed by the invention. Organoid units can be derived from tissues including, but not limited to, spleen, lung, liver, kidney, pancreas, endocrine tissue, heart, esophagus, colon, stomach, gall bladder and uterus. The resulting engineered tissue can comprise spleen, lung, liver, kidney, pancreas, endocrine, cardiac muscle, esophagus, colon, stomach, gall bladder or uterus. The invention further relates to a tissue-engineered organ or organ portion or specific section thereof comprising compact tissue grown in a biocompatible polymer scaffold, wherein the tissue is derived from spleen, lung, liver, kidney, pancreas, endocrine, heart, esophagus, colon, stomach, gall bladder or uterus.

### RELATED APPLICATIONS/PATENTS & INCORPORATION BY REFERENCE

This application claims priority to U.S. application Serial No. 60/291,273, filed on May 16, 2001 and to U.S. application Serial No. 60/343,676, filed on Dec. 27, 2001.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### STATEMENT OF RIGHTS TO INVENTION UNDER FEDERALLY SPONSORED RESEARCH

The United States government may have certain rights in this invention by virtue of grant number No. DAMD17-99-2-9001 from the Department of Defense.

### FIELD OF THE INVENTION

The present invention relates to compositions comprising tissue-engineered organs and portions or specific sections thereof, such as spleen, lung, liver, kidney, pancreas, endocrine glands, cardiac muscle, esophagus, colon, stomach, gall bladder, duodenum, jejunum, and ileum, and methods for the production thereof.

### BACKGROUND

Organ failure or tissue loss is a frequent, life-threatening and costly health problem that has no complete solution to date. Although current interventions, including organ transplantation, surgical reconstruction, mechanical device substitution and metabolic product supplementation, have advanced patient care, each has inherent limitations. For a detailed review on these medical strategies, see Lalan, S. et al. (2001) World J. Surg. 25:1458-1466. New solutions that can overcome these limitations are, therefore, required to promote improved quality of life for many people.

Organ transplantation is a costly procedure that is dependent on donor organ availability, of which there is a chronic shortage, and is complicated by the possibility of host rejection. To prevent host rejection, organ transplantation is accompanied by the lifelong administration of immunosuppressive drugs having very serious side effects, including heart disease, kidney failure and diabetes.

Surgical reconstruction is the substitution of different tissues for a diseased tissue or organ (e.g., using a patient's colon to replace esophagus). Frequently, the results are sub-optimal, with the substituted tissue unable to completely restore lost function of the diseased organ. Improper or limited function of the afflicted organ results in risk of organ morbidity.

Implantable mechanical devices cause concern in that they generally lack biocompatibility, which can result in infection, irritation, inflammation and toxicity.

Furthermore, the durability of the materials is of issue. In specific cases, such as pediatric patients, repeated surgery may be required to adapt to the growth of the patient.

Metabolic product supplementation, wherein hormones are chronically administered, is limited by the notable absence of the normal regulatory feedback mechanisms present in the body to balance the hormones. An imbalance of hormones may result, creating serious thyroid, parathyroid, adrenal and pancreas disorders, including diabetes and osteoporosis.

Multiple tissue losses mandate replacement. For example, the absence of native large intestine, where short chain fatty acid production, sodium and water absorption and storage occur, has been linked to significant morbidity. Post-colectomy morbidity rates, ranging from 5-30%, include important changes in enterohepatic circulation, microbiology, production of short chain fatty acids, storage capacity, and water and sodium absorption (Papa et al. (1997) JACS 184:269-272). The surgical substitution of an ileal pouch for the absent colon has a number of serious complications as well, including severe pouchitis, high stool frequency, megapouch, and pelvic sepsis due to pouch leak (Fonkalsrud, E. W. and Bustorff-Silva, J. (1999) Ann. Surg. 229:197-204). The cumulative frequency of pouchitis at major referral centers approaches 50% over 10 years (Stahlberg et al. (1996) Dis. Colon Rectum 29:1012-1018; Meagher et al. (1998) Br. J. Surg. 85:800-803). Therapies directed to relieving pouch morbidity include revision or conversion to end-ileostomy, transrectal catheter drainage, antibiotics, and steroids (Dayton, M. T. (2000) Amer. Jour. Surg. 180(6):561-565). Ileal mucosal adaptation in the pouch through colonic metaplasia and the presence of chronic inflammation have been postulated to increase long-term neoplasia (Shepherd, N. A. (1990) BMJ 301:886-887).

In addition, post-splenectomy sepsis, which can quickly induce a coma and death, has a mortality rate of up to 50%. This is not surprising, given the critical function of the spleen in the immune response to blood-born antigens. Current splenectomy surgical approaches are haphazard and unstandardized. A common approach is to take 3 mm slices of remaining spleen and implant them into the omentum. Human variability in the process makes the outcome difficult to assess. Furthermore, it has been described in virtually all reports of this approach that the majority of the implanted spleen undergoes necrosis.

Methods of tissue engineering for the generation of replacement tissues can include, for example, the generation of intestinal organoids (Marler, J. et al. (1998) Adv. Drug. Del. Rev. 33:166-182; Choi, R. et al. (1997) Trans. Proc. 29:848-851; Kim et al., (1999) J. of Surg. Res. 87:6-13; Kaihara et al. (1999) Trans. Proc. 31:661-662; Kim et al., (1999) Trans. Proc. 31:657-660; Kaihara et al., (1999) Trans. 67:241-245; Kim et al., (1999) 67:227-233). These studies describe the seeding of intestinal organoid units into polymers followed by transplantation into host intestine, however, failure to achieve high density seeding resulted in thin, sub-optimal engineered tissue. Overcoming this defect is necessary for the production of engineered tissues that function optimally in a wide range of organ systems, such as spleen, lung, liver, kidney, pancreas, endocrine, cardiac muscle, esophagus, colon, stomach, gall bladder or uterus.

Given the limitations of all of the current strategies to treat organ failure or tissue loss, it would be highly desirable to develop engineered tissues that restore, replace, maintain or improve the function of human tissues or organs.

### SUMMARY OF THE INVENTION

The present invention overcomes defects in the prior art through methods achieving high density seeding of polymer scaffolds with organoid units. The present methods advantageously produce compact tissue-engineered compositions, having improved durability and functionality. In addition, the methods and compositions described herein provide an unexpected increase in long-term post-transplantation success rates, due to increased viability of the engineered tissues in a host environment. The methods of the invention are superior to prior art methods in that the engineered tissue comprises an improved anatomic recapitulation of native tissue.

The present invention relates to a method for producing a tissue-engineered organ or portion or specific section thereof, comprising the steps of loading organoid units into a biocompatible polymer scaffold at high density and implanting the tissue-polymer scaffold into a subject.

Organs produced by the methods described herein are also encompassed by the present invention.

The invention further provides a method for producing tissue-engineered duodenum, jejunum or ileum comprising the steps of:

(a) preparing organoid units by

(i) obtaining pieces of duodenal tissue to engineer duodenum, pieces of jejunal tissue to engineer jejunum or pieces of ileal tissue to engineer ileum;

(ii) digesting the tissue obtained in (i) with an enzyme;

(iii) purifying the tissue to obtain organoid units;

(b) loading the organoid units into a biocompatible polymer scaffold at a high density; and

(c) implanting the polymer scaffold into a subject.

The invention also relates to a tissue-engineered organ or portion or specific section thereof comprising compact tissue and a biocompatible polymer scaffold, wherein the tissue is derived from sources including but not limited to spleen, lung, liver, kidney, pancreas, endocrine, heart, esophagus, colon, stomach, gall bladder or uterus. The tissue-engineered organ or portion or section thereof can comprise spleen, lung, liver, kidney, pancreas, endocrine, cardiac muscle, esophagus, colon, stomach, gall bladder or uterus and preferably, is of the same tissue type as the organoid unit from which it is produced.

These and other objects and embodiments are described in or are obvious from the following Detailed Description and are within the scope of the invention.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference and described as follows.

FIG. 1 shows a polymer scaffold.

FIG. 2 shows organoid units from colon, labeled with fluorescent dye (20×) that were then implanted in the omentum (Top: Fluorescence off; Bottom: Fluorescence on).

FIG. 3 shows a representative section of an intact large intestine taken from an ITEC animal at Day 41.

FIG. 4 shows a surgical construct harvested from ITEC animals.

FIG. 5 shows weight loss in ITEC and IL animals.

FIG. 6 shows stool transit times in ITEC and IL animals.

FIG. 7 shows dry stool weight/wet stool weight in ITEC and IL animals.

FIG. 8 shows total serum bile acids in ITEC and IL animals.

FIG. 9 shows serum cholic acid in ITEC and IL animals.

FIG. 10 shows serum sodium in ITEC, IL, and unoperated animals.

FIG. 11 shows stool short chain fatty acids in ITEC and IL animals.

FIG. 12 shows stool n-butyrate levels in ITEC and IL animals.

FIG. 13 shows stool short chain fatty acid distribution in ITEC and IL animals.

FIG. 14 shows a representative tissue-engineered colon cyst after four weeks in the omentum.

FIG. 15 shows a tissue-engineered colon cyst after four weeks in the omentum with the lumen exposed.

FIG. 16 shows H&E staining of tissue-engineered colon.

FIG. 17 shows periodic acid-Schiff (PAS) staining of tissue-engineered colon.

FIG. 18 shows trichrome staining of tissue-engineered colon (top: native colon; bottom: TEC).

FIG. 19 shows smooth muscle actin staining of tissue-engineered colon (A: native colon, B: tissue-engineered colon).

FIG. 20 shows S100 staining of tissue-engineered colon (A: native colon, B: tissue-engineered colon).

FIG. 21 shows large ganglion cells in S100 positive cells of tissue-engineered colon (A: native colon, B: tissue-engineered colon).

FIG. 22 shows a TUNEL assay (top: native colon; bottom: tissue-engineered colon).

FIG. 23 shows anti-acetylcholinesterase staining of tissue-engineered colon.

FIG. 24 shows transmission electron microscopy photographs of tight junctions, abundant mitochondria and desmosomes with keratin filaments in tissue-engineered colon.

FIG. 25 shows transmission electron microscopy photographs of apical microvilli in tissue-engineered colon.

FIG. 26 shows transmission electron microscopy photographs of neuroendocrine cell and goblet cell in tissue-engineered colon.

FIG. 27 shows H&E staining of Tissue Engineered Esophagus Derived from Adult Organoid Units (10×).

FIG. 28 shows organoid units from esophagus, labeled with green fluorescent protein that were then implanted in the omentum (top: fluorescence off; bottom: fluorescence on).

FIG. 29 shows tissue-engineered esophagus stained for actin, indicative of smooth muscle, which is seen in its normal location (top: native esophagus; bottom: tissue-engineered esophagus).

FIG. 30 shows tissue-engineered esophagus after 4 weeks of growth seen at 20× magnification.

FIG. 31 shows a fluoroscopic picture of tissue-engineered esophagus anastomosed to native esophagus (top: native esophagus; bottom: tissue-engineered esophagus).

FIG. 32 shows H&E staining of Tissue Engineered esophagus after living as interposition graft.

FIG. 33 shows tissue-engineered spleen at 4 weeks.

FIG. 34 shows spleen von Willebrand factor staining, 20× magnification (top: native spleen; bottom: tissue-engineered spleen).

FIG. 35 shows spleen CD3 staining, 20× magnification (top: native spleen; bottom: tissue-engineered spleen).

FIG. 36 shows organoid units from small intestine, labeled with green fluorescent protein that were then implanted in the omentum (top: fluorescence off; bottom: fluorescence on).

FIG. 37 shows H&E staining (10× magnification) of tissue-engineered small intestine section three weeks after implantation (top: native small intestine; bottom: tissue-engineered small intestine).

FIG. 38 shows organoid units from pancreas, labeled with green fluorescent protein that were then implanted in the omentum (top: fluorescence off; bottom: fluorescence on).

FIG. 39 shows tissue-engineered pancreas (top: 10× magnification; bottom: 20× magnification).

FIG. 40 shows H&E staining (10× magnification) of tissue-engineered pancreas revealing loose organization seen with surrounding fibrosis.

FIG. 41 shows tissue-engineered pancreas ducts (10× magnification); ductular proliferation of tissue-engineered pancreas (top: 20× magnification; Middle: 40× magnification; bottom 60× magnification).

FIG. 42 shows “islet ball” in tissue-engineered pancreas (20× magnification).

FIG. 43 shows H&E staining of tissue-engineered pancreas revealing acinic structures (top: 10× magnification; bottom: 20× magnification).

FIG. 44 shows tissue-engineered pancreas stained for insulin (top: 20× magnification; bottom: 40× magnification).

FIG. 45 shows tissue-engineered pancreas stained for glucagon (top: 10× magnification; bottom 40× magnification).

FIG. 46 shows organoid units from stomach, labeled with green fluorescent protein that were then implanted in the omentum (top: fluorescence off; bottom: fluorescence on).

FIG. 47 shows H&E stained Tissue Engineered stomach (10×).

FIG. 48 shows H&E stained Tissue Engineered stomach (20×).

FIG. 49 shows H&E stained anastomosed Tissue Engineered Stomach (10×).

FIG. 50 shows immunohistochemical staining for α-actin smooth muscle was positive in the stroma adjacent to the neomucosa, confirming the presence of smooth muscle layers (10×).

FIG. 51 shows immunohistochemical staining for gastric mucin was positive on the surface of the gastric epithelium, indicating a well developed gastric epithelium (20×).

FIG. 52 shows immunohistochemical staining for gastrin was positive, indicating intact APUD (Amino Precursor Uptake and Decarboxylase) cells (Top:20×; Bottom:40×).

### DETAILED DESCRIPTION

The present methods comprise the use of a biocompatible polymer scaffold for the seeding and attachment of organoid units in vitro, followed by implantation of functional engineered tissue into patients. The result is a tissue-engineered organ or a portion or specific section thereof, which is vascularized in vivo, to support cell growth in a three-dimensional configuration. The design and construction of the polymer scaffold, as well as the conditions and methods of organoid unit isolation and seeding, advantageously produce compact engineered tissue compositions having a high rate of success post transplantation.

The terms “comprises”, “comprising”, and the like, can have the meaning ascribed to them in U.S. Patent Law and can mean “includes”, “including” and the like.

In the context of the present invention, an “organoid unit” comprises a cluster of isolated tissue comprising all of the cell types found in a cross-section of the native tissue, including organ-specific stem cells. Preferably, organoid units comprise mesenchymal cores surrounded by epithelium. Preferably, organoid units derived from heart comprise all cells present in a cross section from the full thickness of heart tissue, including but not limited to cardiac muscle cells, nerve cells, vascular cells and mesenchymal cells.

In the context of this application, organoid units seeded on a biocompatible polymer scaffold are referred to as an “organoid unit composition.” Organoid units can be derived from tissues including, but not limited to, spleen, lung, liver, kidney, pancreas, endocrine tissue, heart, esophagus, colon, stomach, gall bladder and uterus. Examples of endocrine tissues include, but are not limited to, thyroid, parathyroid, pituitary, hypothalamus, gonads, salivary and adrenal glands. The resulting engineered tissue can likewise comprise spleen, lung, liver, kidney, pancreas, endocrine, cardiac muscle, esophagus, colon, stomach, gall bladder or uterus, and preferably, is of the same tissue type as the organoid unit from which it is produced. For example, organoid units derived from spleen can produce spleen, organoid units derived from liver can produce liver, organoid units derived from pancreas can produce pancreas, and organoid units derived from endocrine tissue can produce endocrine glands, and so forth.

As referred to herein, a “solid organ” is an organ selected from the group consisting of spleen, lung, liver, kidney, pancreas, heart, colon, gall bladder and uterus.

In a preferred embodiment, a homogenous organoid unit composition derived from a single tissue source is implanted into a host. In another embodiment, heterogeneous organoid unit compositions derived from multiple tissue sources are implanted into a host. Tissue from which organoid units are derived can be harvested from a neonatal, juvenile, or adult donor, or even from previously engineered tissue. The transplantation can be autologous, such that the donor of the tissue from which organoid units are derived is the recipient of the engineered-tissue. The transplantation can be heterologous, such that the donor of the tissue from which organoid units are derived is not that of the recipient of the engineered-tissue.

Organoid units are loaded into the polymer scaffold at a high density. Preferably, between about 100,000 and 1,000,000 organoid units per 1.5 cm² of polymer are loaded. More preferably, at least about 100,000 organoid units per 1.5 cm² of polymer are loaded. Polymer scaffold density in this range advantageously saturates the scaffold, promoting formation of compact engineered-tissue having an improved success rate post transplantation as compared to prior art methods. For purposes of this invention, “compact engineered-tissue” is a continuous laminar structure with sufficient density such that there are no bare areas in the engineered tissue. Compact tissue also has sufficient cell-cell and cell-matrix attachments such that it does not disintegrate.

Engrafted organoid unit compositions can assimilate within a variety of host tissues, including spleen, lung, liver, kidney, pancreas, endocrine, cardiac muscle, esophagus, colon, stomach, gall bladder, uterus, duodenum, jejunum, and ileum. Once transferred into a host, the tissue-engineered organs recapitulate the function and architecture of the native host tissue. The tissue-engineered organs will benefit patients in a wide variety of applications, including the treatment of cancer, congenital defects, or damage due to surgical resection.

In another aspect of the present invention, the engineered-tissue is functionally optimized for a particular anatomical section within an organ. For example, the stomach has many functions, some of which are specific to distinct anatomical sections, such as the fundus, corpus or antrum. Secretion of HCl from parietal cells, gastrin from G cells, and pepsinogen from chief cells all occur via different stimuli. A gastrin-producing tissue-engineered stomach can be efficiently produced from organoid units comprising tissue derived from the same. For example, pyloric glands can be harvested from the antrum and/or pyloric regions of the stomach and implanted in a host as part of an organoid unit composition, resulting in a tissue-engineered stomach that produces gastrin. Similarly, oxyntic glands of the fundus and corpus can be implanted in a host as part of an organoid unit composition, resulting in a tissue-engineered stomach that secretes HCl as well as Intestinal Factor, which is necessary for absorption of vitamin B12 by the ileum of the small intestine. Alternatively, a specific section of ileum can also be harvested and implanted as part of an organoid unit composition to produce a tissue-engineered small intestine in which B12 is absorbed.

Organoid units can be derived from different organs, such as small and large intestine, to produce engineered-tissue having multiple desired properties. For example, organoid units derived from small and large intestine have general absorption properties of small intestine, but the water absorption and hardiness of large intestine.

Other examples of organs comprising specific regions in which engineered-tissue can be implanted or alternatively used as source material include, but are not limited to, the renal cortex or the medulla of the kidney, the duodenum (important properties include the release of cholecystokinin and secretin and iron absorption), ileum (important properties include bile and salt uptake) and jejunum of the small intestine, and the regions of the colon (important properties include gradients for aldosterone mediated sodium and water absorption, potassium secretion and short chain fatty acid fermentation). Other known tissue-specific locations of particular functions within organs are listed in standard physiology textbooks (see, for example, Guyton and Hall, “Textbook of Medical Physiology, 10^{th} ed.” (2000) W. B. Saunders Co.) and are within the scope of the invention.

The following principals guide tissue-engineered compositions and methods of the present invention:

1. Every structure in living organisms is in a dynamic state of equilibrium, undergoing constant renewal, remodeling and replacement of functional tissue, which varies from organ to organ and structure to structure. Organoid units dissociated from their native environment will reform tissue-specific structures, the extent to which will depend on the environment in which they are placed. For example, organoid unit compositions derived from native liver will develop liver specific structure and function to the greatest extent upon transplantation into host liver.

2. Non-vascularized engineered-tissue generally cannot be implanted in volumes greater than about between one to three mm³, because nutrition is supplied by diffusion until new blood vessels form, and this distance is the maximum distance over which diffusion can transpire prior to angiogenesis.

3. Cell shape is determined by cytoskeletal components; attachment to matrix plays an important role in cell division and differentiated function. If organoid unit compositions are placed into mature tissue as a suspension without cell attachment provided by way of a polymer scaffold, formation of attachment sites, achieving polarity and overall functioning is hindered for lack of intrinsic organization. The matrices must have sufficient surface area and exposure to nutrients, such that cellular growth and differentiation can occur prior to the ingrowth of blood vessels following implantation. After implantation, the configuration must allow for diffusion of nutrients and waste products and for continued blood vessel ingrowth as cell proliferation occurs.

Polymer scaffolds of the present invention function in place of a connective tissue scaffold or matrix, and are designed to optimize gas, nutrient, and waste exchange by diffusion until neovascularization occurs from the host environment. Polymer scaffolds comprise, for example, a porous, non-woven array of fibers. They preferably comprise polyglycolic acid (PGA), tubular in shape.

The design and construction of the polymer scaffold is of primary importance. The polymer scaffold can be shaped to maximize surface area, to allow adequate diffusion of nutrients and growth factors to the cells. The maximum distance over which adequate diffusion through densely packed cells can occur is in the range of about 100 to 300 microns (μm), under conditions similar to those which occur in the body, wherein nutrients and oxygen diffuse from blood vessels moving into the surrounding tissue. (See, e.g., Vacanti et al. 1998, U.S. Pat. No. 5,759,830). Taking these parameters into consideration, one of skill in the art would configure a polymer scaffold having sufficient surface area for the cells to be nourished by diffusion until new blood vessels interdigitate the implanted engineered-tissue using methods known in the art. Preferably, the polymer scaffold comprises a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation. Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, teflon RTM, nylon silicon, and shape memory materials, such as poly(styrene-block-butadiene), polynorbomene, hydrogels, metallic alloys, and oligo(ε-caprolactone)diol as switching segment/oligo(p-dioxyanone)diol as physical crosslink. Other suitable polymers can be obtained by reference to The Polymer Handbook, 3rd edition (Wiley, N.Y., 1989).

In a preferred embodiment, polymer scaffolds are made of synthetic, highly porous, biodegradable, non-woven sheets of PGA (Smith and Nephew, Heslington, York, United Kingdom). “Highly porous” means at least 95% porous. A stenting shape, such as a tube, that keeps a lumen open rather than allowing it to collapse, thus making multiple small discontinuous cysts, is preferred. Length of the tubular structure can be in the range of about 1-7 cm; outer diameter can be in the range of about 0.5-10 mm. One of skill in the art can readily vary the parameters of size and shape to suit human and non-human hosts. Preferably approximately 5% poly-L-lactic acid is applied to the surface of the PGA. Factors, including nutrients, growth factors, inducers of differentiation or de-differentiation, products of secretion, immunomodulators, inhibitors of inflammation, regression factors, biologically active compounds which enhance or allow ingrowth of the lymphatic network or nerve fibers, and drugs, can be incorporated into or provided in conjunction with the polymer scaffold. In another embodiment, attachment of organoid units to the polymer scaffold is enhanced by coating the scaffold with compounds such as basement membrane components, agar, agarose, gelatin, gum arabic, collagens types I, II, III, IV, and V, fibronectin, laminin, glycosamino-glycans, mixtures thereof, and other materials known to those skilled in the art.

In another embodiment, cells at the site of implantation are exposed to a biodegradable polymer that includes “de-differentiators,” which are compounds that induce a reversion of the surrounding mesenchymal cells such that they become de-differentiated, or embryonic. The-implanted organoid unit composition may then develop better, for example, in a fetal environment, than it would if surrounded by more mature cells.

In a preferred embodiment, organoid units are prepared as described in Example 1.

Briefly, tissue is harvested from a subject, ravaged, cut into pieces, and washed. A “host,” “patient” or “subject” is a vertebrate, preferably a mammal, and most preferably a human. Mammals include, but are not limited to, humans, farm animals, sport animals, and pets. One of skill in the art can readily vary the parameters of the methods described herein to accommodate hosts or subjects of variable size and species, including but not limited to, humans of any age.

The tissue can be minced mechanically, or more preferably, digested with an enzyme or enzyme mixture. Any digestive enzyme known in the art may be used, including, but not limited to, proteases, dispase, collagenase, trypsin and chymotrypsin. Conditions and duration of enzymatic digestion will vary, depending upon the type of tissue and enzyme or enzyme mixture used, and would be able to be determined by one skilled in the art. For example, the preferable enzyme mixture is dispase/collagenase, and digestion is ideally performed at approximately 37° C. for about 20-35 minutes. Following mincing or digestion, organoid units are purified by washing and centrifugation, prior to reconstitution in media and seeding on polymer scaffolds.

The washing steps are particularly important in the preparation protocol; thorough washing is required to obtain clean organoid units. Organoid units prepared under this procedure attach to the polymer scaffold better than those of the prior art, resulting in a greater density of tissue at implantation, larger cysts, and compact tissue in the engineered organ.

Purified organoid units are seeded or loaded onto polymer scaffolds with a pipette. Preferably, the pipette is first rinsed with high glucose DMEM with about 5-10% inactivated fetal bovine serum to prevent sticking of organoid units to the inner walls of the pipette. Organoid units are loaded into the hollow center of the polymer scaffold and are dripped over the top so that the scaffold is saturated with the organoid units. Seeding is preferably performed on ice.

Prior to seeding on polymer scaffolds, organoid units can be marked by transfection with a plasmid expressing Green Fluorescent Protein (GFP). Organoid units transfected with such a vector express GFP, providing a tool for identifying native tissue versus implanted tissue. Native tissue generally does not autofluoresce, which can be confirmed for the specific tissue in question prior to fluorescence studies; therefore, any fluorescence observed in examination of engineered organs or portions or specific sections thereof is the result of implanted organoid unit compositions. A GFP marker is useful to evaluate long term population of engineered-tissue, and is not due to host repopulation of a suboptimal graft. Red Fluorescent Protein (RFP) can also be used in the same way as GFP, and other dyes, for example, Sigma red, can be used as cell markers as well.

Tissue-engineered compositions of the present invention have a number of advantages over either pharmacological manipulation or organ transplantation for replacing or supplementing lost organ function. Although great strides have been made in these areas, presently available means for tissue replacement or supplementation are often deficient. Success in transplantation or pharmacological manipulation may modify the outcome of a disease, but it usually does not result in cure, or it trades the original disease for the complications of non-specific immunosuppression.

Thus, one advantage of the present method is that it provides a means for selective transplantation of tissue that possesses the necessary biological function(s), without transplantation of passenger leukocytes and antigen-presenting cells. The result is greatly reduced risk of rejection of tissue without the use of drugs, especially if one is able to implant organoid units derived from the same or similar human leukocyte antigen (HLA) tissue type.

The prospect of implanting an organoid unit composition derived from the recipient's own tissue has a further, more fundamental advantage: the elimination of the need for organ donors. For example, if a patient has lost a percentage of his or her intestine because of ischemic damage, tissue from the remaining part of the intestine can be harvested, seeded onto the appropriate polymer scaffold, and placed back into the patient, to be allowed to vascularize, grow and function as a neointestine.

In the case of replacement of liver function, it may be possible to construct a cell-matrix structure from a small piece of liver. It is thought that only about 10% of hepatic cell mass is necessary for adequate function. This makes implantation of organoid unit compositions into children especially preferable to whole organ transplantation, due to the relatively limited availability of donors and smaller size of juvenile organs. For example, an 8-month-old child has a normal liver that weighs approximately 250 g. That child would therefore need about 25 g of tissue from a biopsy a compatible donor. An adult liver weighs-approximately 1500 g; therefore, the biopsy would only be about 1.5% of the adult liver. When organoid units are attached to the polymer scaffold with high efficiency and implanted, proliferation in the new host will occur, and the resulting hepatic cell mass replaces needed function.

The invention shall be further described by the following examples, which are provided to illustrate the invention, and should not be considered to limit the scope in any way.

### EXAMPLES

### Example 1

### Tissue-Engineered Colon Derived from Neonatal Tissue Exhibits Function In Vivo

In vivo colon replacement by tissue-engineered colon (TEC) in lieu of an ileal pouch was performed. End ileostomies were created in 22 male Lewis rats. In 11 animals, side-to-side ileum-TEC anastomosis was performed 1 cm from the stoma (ITEC). This group was compared to end-ileostomy alone (IL). Serial weights were measured, and animals were harvested sequentially for assessment of histological signs of pouchitis. Transit times, stool dry and wet weights, and serum and stool colon function markers were collected. Animals survived to 41 days. Weight loss was more than 1.5 times greater in IL animals compared to ITEC animals. The transit times of ITEC animals were significantly longer than in IL animals, with lower stool moisture content and higher total serum bile acids. Animals without tissue-engineered colon had statistically significant hyponatremia, elevated Blood Urea Nitrogen (BUN) levels, and lower stool short chain fatty acids (13.5 μmole/kg in animals without tissue-engineered colon compared to 84.2 μmol/kg in animals with tissue-engineered colon) with an abnormal distribution. The tissue-engineered colon successfully recapitulated several major physiologic functions of native large intestine in vivo.

Organoid Unit Preparation Colon organoid units were produced by dissecting the sigmoid colon without mesentery from four-day-old Lewis rat pups (n=40), although the organoid units can be generated from adult as well as neonatal rats, so the donor age is not critical. Colon was irrigated with cold Hanks Balanced Salt Solution (HBSS; Cellgro) without calcium or magnesium to clean luminal contents, then cut into full thickness 2 mm×2 mm sections after lengthwise opening along the antimesenteric border. These were washed three times in 4° C. HBSS, sedimenting between washes, and digested with dispase (Boehringer Mannheim) 0.25 mg/mL and collagenase (Worthington Biochemical Corporation) 800 U/mL on an orbital shaker at 37° C. for 20 minutes. The collagenase activity is lot dependent and anyone skilled in the art would know how to titrate a specific batch to determine optimal working conditions. The digestion was immediately stopped with three 4° C. washes of a solution of high glucose Dulbecco's Modified Eagle Medium (DMEM; Gibco), 4% heat-inactivated fetal bovine serum (iFBS), and 4% sorbitol. The organoid units were centrifuged between washes at 150 rpm for five minutes, and the supernatant removed. Organoid units were reconstituted in high glucose DMEM with 10% iFBS, counted by hemocytometer, and loaded with approximately 100,000 units per polymer at 4° C., maintained at that temperature until implantation.

Polymer Scaffolds

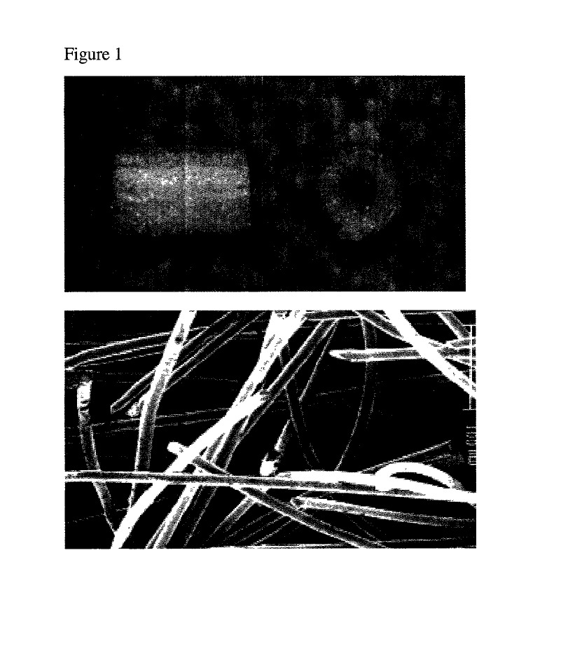

Polymer scaffolds (FIG. 1) were constructed of 2 mm thick nonwoven polyglycolic acid, formed into 1 cm tubes (Outside Diameter (OD)=0.5 cm, Inside Diameter (ID)=0.2 cm), and sealed with 5% Poly (L-Lactic acid). Polymer tubes were sterilized in 100% ethanol for twenty minutes, then washed with 500 mL of phosphate buffered saline (PBS), coated with 1:100 collagen Type 1 PBS solution for twenty minutes, and washed again with 500 mL PBS. Polymer tubes were internally loaded with organoid units by micropipette.

Implantation of Tissue-Engineered Colon into Rat Omentum

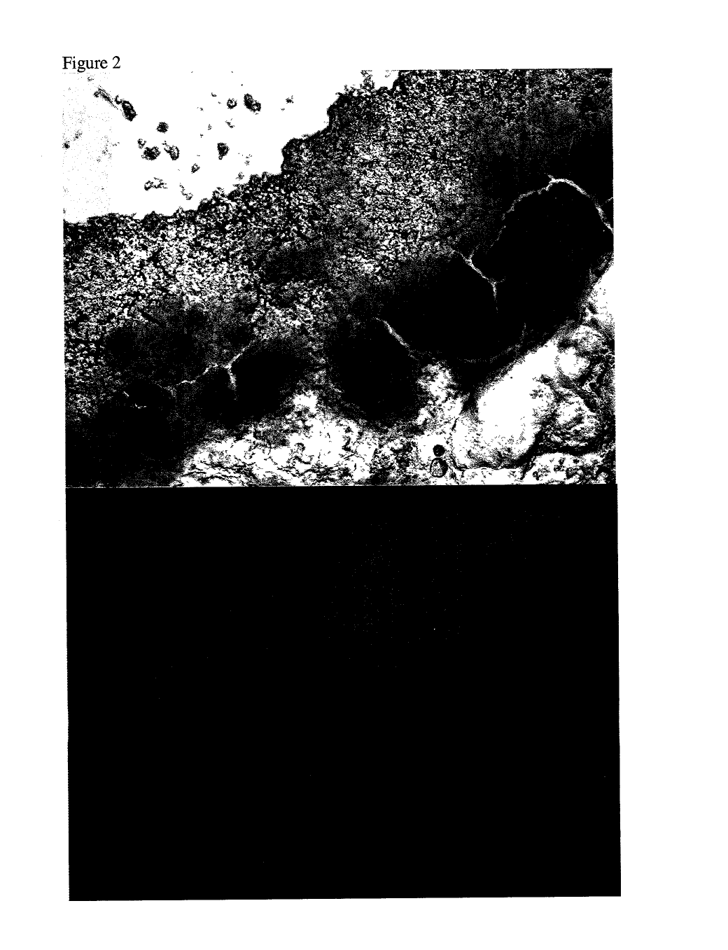

Under pentobarbital anesthesia, 150 g Lewis rats were either implanted with tissue-engineered colon (TEC) constructs (group ITEC, n=11) or subject to sham laparotomy (control group IL, n=11). Implantation was achieved through a 1 cm upper abdominal incision just under the xiphoid, through which the greater omentum was externalized and wrapped around the TEC construct, secured with a 6-0 prolene suture. Essentially the omentum was brought out without any tearing through the small incision and laid out flat. The top of the omentum was extended down the length of the polymer so that it was completely covered. The bottom of the omentum was then extended to the top of the omentum so that the polymer was completely covered twice. The side of the omentum was wrapped over the two previous layers so that the polymer was enfolded at the point at which all the layers overlap. The stitch minimally incorporated the polymer. The polymer/omentum construct was returned to the peritoneum, with care not to torse the pedicle, laying the polymer away from the liver to avoid adhesions to the liver, before closing the abdomen in layers with 3-0 vicryl or similar suture. The implantation was also performed with colon organoid units that had been labeled with GFP for one hour at 37° C. to distinguish and verify engineered tissue from host tissue (FIG. 2). Confirmation of GFP production was obtained by simultaneous in vitro culture of the organoid units. Four weeks later, end ileostomies were created in all 22 rats. In ITEC animals, side-to-side ileum-TEC anastomosis was performed 1 cm from the stoma after measuring the dimensions of the TEC.

Sample Protocol for Derivation of Engineered Tissues

The examples presented herein were generated using an in vitro rat model system in which tissue engineering is reasonably correlated to tissue engineering in humans. Below is a sample protocol for the derivation and implantation of tissue-engineered constructs derived from multiple tissue sources (e.g., liver, spleen, colon, esophagus, heart, pancreas). The tissue source can be neonatal, adult or even engineered tissue. The parameters described below can be extrapolated to tissues derived from and/or transferred to human hosts.

1. Cysts can be generated from adult as well as neonatal subjects; the donor age is not critical.

2. Tissues are harvested using sterile technique. Colon is harvested excluding any mesenteric attachments. Spleen is removed by standard splenectomy. Pancreas is removed by standard pancreatectomy. Harvested tissue is placed in HBSS (Cellgro catalog number 21-021-CV) without calcium or magnesium at 4° C. For a harvested colon, the lumen is taken after the cecum and just proximal to the rectum is intubated with an angiocatheter and washed with cold HBSS once, with visual confirmation that all fecal material is cleared. Specific tissue sources within an organ can be surgically removed for use in generating tissue-engineered constructs, e.g. renal cortex and medulla of the kidney, fundus, corpus and antrum of the stomach, and the duodenum and ileum of the small intestine, or combinations thereof to generate chimeric engineered tissue. These constructs can then be implanted at their native organ site or in other organ sites, thereby creating chimeric organs.

3. Tissues are transferred to clean HBSS. Gastrointestinal tissues are opened longitudinally with iris scissors and then cut into 1 mm pieces. Spleen/pancreas is washed in HBSS and clipped into 2 mm pieces. All tissues are kept separate with separate instrumentation.

4. The tissue mix is transferred to a 50 ml Falcon tube and the material is allowed to precipitate for 2-3 minutes.

5. At least two washes of cold HBSS are performed (or until the HBSS retains its red clarity, less for duodenum, more for small intestine (e.g., up to 20 washes) or higher amounts of tissue). For colon, small pieces of tissue are allowed to re-settle to the bottom of the tube during each wash for approximately 3 minutes. There is no trituration. Natural vortexing occurs from the wash of HBSS each time. Solid organs (e.g. spleen or pancreas) are washed twice to remove blood or contaminants. When the HBSS retains its red clarity, the solution is removed in a sterile fashion once more when the material has precipitated to the bottom of the tube.

6. Enzyme solution is sterile filtered in a quantity of 100 mL HBSS comprising: 10 mg of Dispase from Bacillus polymyxa (Boehringer Mannheim 14959800) and 3.4 mg of collagenase type 2 (Worthington biochemical corporation 40B3778-A). Solution is made just prior to use and warmed to 37° C. in a water bath. The solution is contacted with the tissue (e.g., 45 cc/vial). This assumes the contents of the vial to be about 5 cc of tissue loosely precipitated. Scale up is carried out proportionally for larger tissue samples.

7. The mix is incubated (37° C.) on a quickly shaking platform for 35 minutes (solid organs such as spleen and pancreas require less time, roughly 20 minutes). The solution should turn brown or tan. It is triturated with a 10 cc pipet for three minutes, or until it flows easily. The total trituration time should be up to ten minutes, indicating proper digestion. Fresh purification solution is applied to the tissue, which has been kept cold. The purification solution is composed of the following: 500 cc High glucose DMEM (Gibco) with 16.4 g of D-sorbitol and 18 cc Fetal Bovine Serum, filtered.

8. Centrifugation is carried out at 400 rpm×4 minutes, followed by removal of supernatant and reapplication of the purification solution with vigorous shaking before centrifugation again at 400 rpm×4 minutes. This step is repeated at least two times.

9. The supernatant is removed and a final solution is applied, comprising 500 cc high glucose DMEM with 10% Fetal Bovine Serum. After centrifugation, the supernatant is again removed and the pellet remains.

10. The organoid units (which are in the pellet) are counted using a hemocytometer.

11. Using a 1 cc plastic pipet, the polymers are then loaded from either end. About 100,000 organoid units are loaded per 1 cm polymer of PGA/Poly (L-Lactic acid) tube. The loading is done in a sterile petri dish on an ice pack, then kept cold on ice for twenty minutes.

12. The subject is anesthetized and a 1.5 cm laparotomy (proportions will vary with the size of the subject) just under the xiphoid is made and the omentum is brought out. For larger subjects, minimally invasive or laproscopic procedures can be used. The polymer is wrapped in the omentum with one 6-0 prolene stitch closing the omentum over the polymer like an envelope. Essentially the omentum is brought out without any tearing through the small incision and laid out flat. The top of the omentum is extended down the length of the polymer so that it is completely covered. The bottom of the omentum is then extended to the top of the omentum so that the polymer is completely covered twice. The side of the omentum is wrapped over the two previous layers so that the polymer is enfolded again. The entire wrap is then turned 90 degrees and one 6-0 Prolene stitch is placed at the point at which all the layers overlap. The stitch minimally incorporates the polymer. Polymers are preferably comprised of 2 mm non-woven polyglycolic acid sealed with 5% Poly (L-Lactic acid), with a length of 1 cm and a fiber density of 15 μm. Porosity is preferably greater than about 95% and the mean pore size is 250 μm.

13. The polymer/omentum construct is returned to the abdomen through the incision with care not to torse the pedicle, laying the polymer away from the liver to avoid adhesions to the liver. In the case of spleen implants, splenectomy can be either performed or avoided.

14. The laparotomy is closed in layers with 3-0 vicryl or similar suture. The subjects are kept comfortable and given a normal diet.

Animals

Animals were housed in mesh bottom cages for stool collection with stoma care daily. All animals were given a normal diet. They were allowed free access to lab chow and water, consuming approximately equal amounts after postoperative day 1.

Statistical Analysis

Statistics were executed by two-stage analysis, two-tailed t-test with Welch correction or ANOVA with Tukey-Kramer multiple comparison test. Means are reported +/−SD unless specified. Commercial laboratory standard errors were reported with the tests.

Results

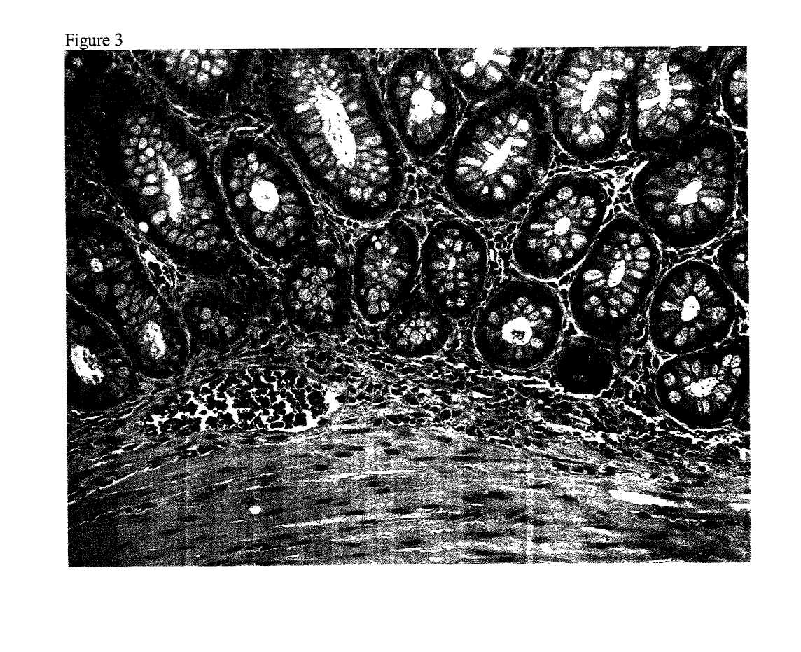

All animals in both groups survived for 41 days at which time they were sacrificed. Formalin-fixed tissue was sectioned and stained with Hematoxylin and eosin (H&E) at days 3, 11, 15, and 20 as well as at day 41 to observe tissue architecture and for evidence of inflammation or pouchitis according to the St. Mark's criteria (Moskowitz et al. (1986) Int. J. Colorectal Dis. 1:167). Hematoxylin and eosin (H&E) staining revealed no evidence of pouchitis in ITEC or IL animals on serially collected tissue. ITEC animal histology revealed an intact large intestine architecture including normal epithelium, vascularization, present ganglion cells, and muscularis propria. FIG. 3 shows a representative section taken at Day 41.

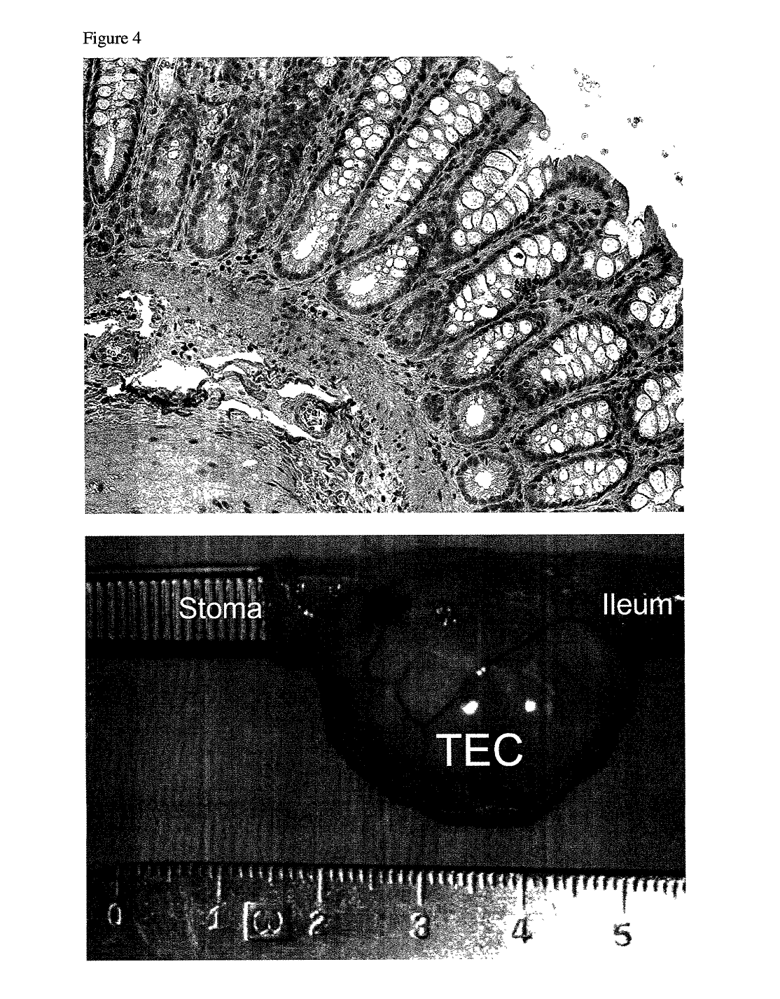

All cysts were spheroids with a fibrous wall and thick mucous distending the lumen. Pouch size was measured at the time of anastomosis and at harvest, and the area calculated with the formula for an oblate spheroid. The average volume was 420+/−98 cubic centimeters. Pouch size on harvest was an average of 6% greater than at the time of anastomosis, with no observation of megapouch. The largest pouch at harvest was 6 cm×4 cm×4 cm. The smallest was 3 cm×2 cm×3 cm (FIG. 4). Assessment of symptoms of pouchitis was negative.

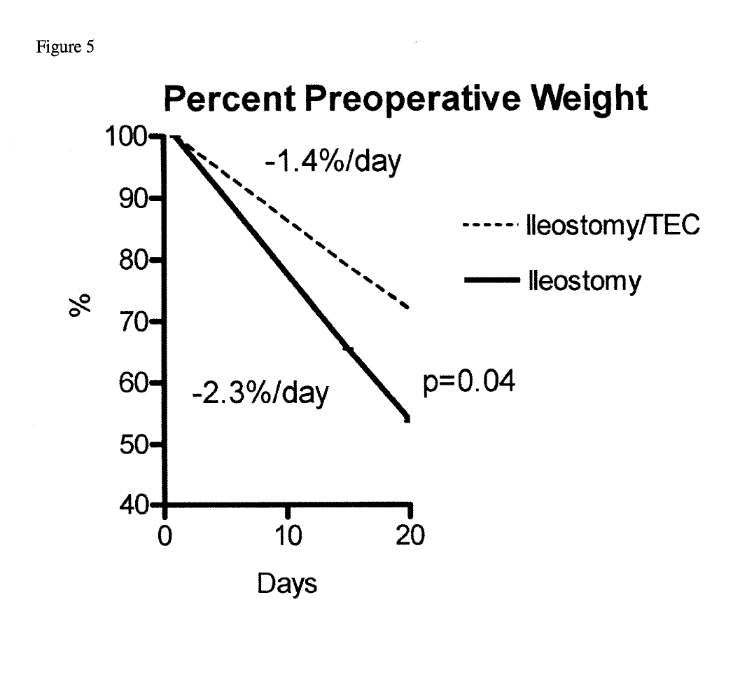

Weights were measured QOD (every other day). Weight loss in the IL animals was statistically significantly greater than ITEC animals with a slope of −2.3% per day (SEM=0.33%) compared to −1.4% per day (SEM=0.29%), p=0.04. Weight loss continued in both groups until day 20-25. By day 30, all animals began to maintain weight. No animals in either group by the time of sacrifice regained weight (FIG. 5).

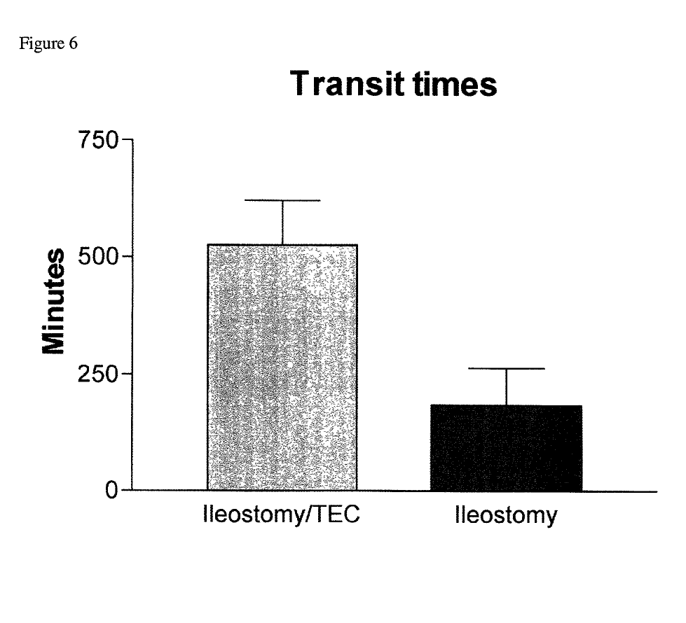

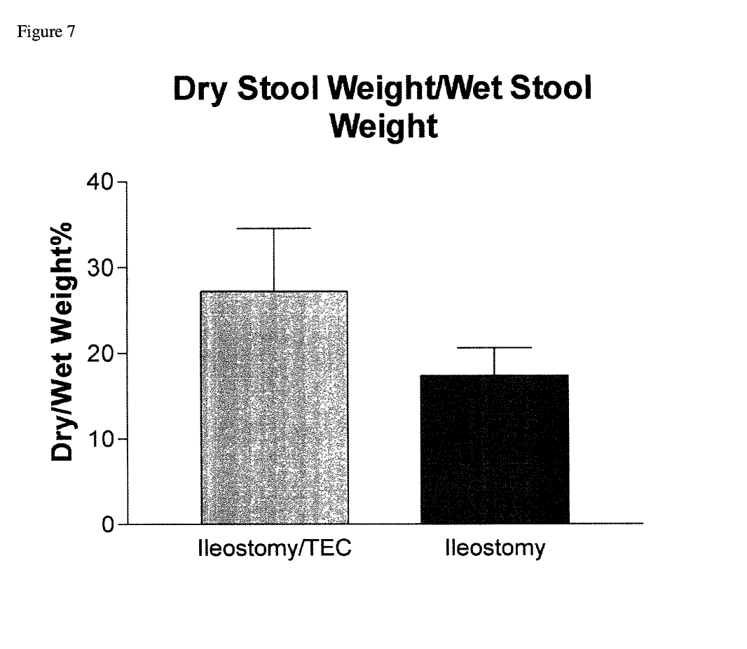

Stool dry and wet weights were recorded on days 19 and 20. Stool from IL and ITEC animals was collected on Day 21 and pooled by group in Cary-Blair Medium and 5% Formalin for analysis of metabolic/absorption markers and microbiology performed by a commercial laboratory. Transit times were measured by phenol red gavage and five minute observations. Stool transit times (FIG. 6) were significantly longer for ITEC animals (525 minutes+/−96) than IL animals (185.5+/−79, p=0.0067). Dry stool weight/wet weight (FIG. 7) was lower in IL animals (17.4%+/−3.2) than ITEC animals (27.2%+/−7.3, p=0.0403). The date on which formed stool was first observed was recorded. The date to formed stool was not statistically significant and varied from day 3-7 for both groups.

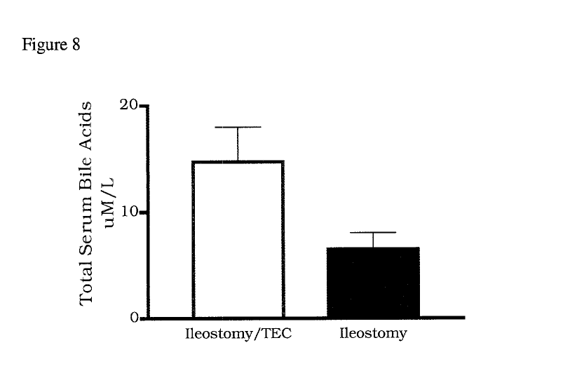

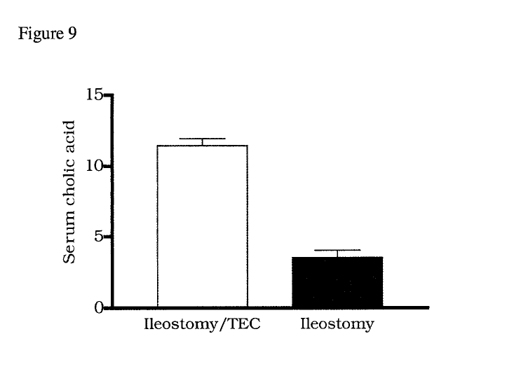

Blood was collected at sacrifice for measurement of electrolytes, lipids, albumin, and bile acids. Normal values were obtained from unoperated matched rats. Total serum bile acids (FIG. 8) were elevated in ITEC animals (74.280 μmole/L+/−40.7) above values observed for IL animals (15.36+/−9.8, p=0.0349). Some of this was accounted for by a statistically significantly increased cholic acid serum content (FIG. 9) in animals with TEC (11.425 μmole/L) versus IL animals (3.550, p=0.0026). There was no statistically significant difference in the levels of deoxycholic acid or chenodeoxycholic acid between the two groups.

Differences in osmolarity, divalents, creatinine, fasting glucose, triglycerides, cholesterol, and globulin were not significant. Albumin was identical in IL (1.075 g/dl) and ITEC (1.35, p=0.0042) animals, although both were lower than rats that had not undergone any operation.

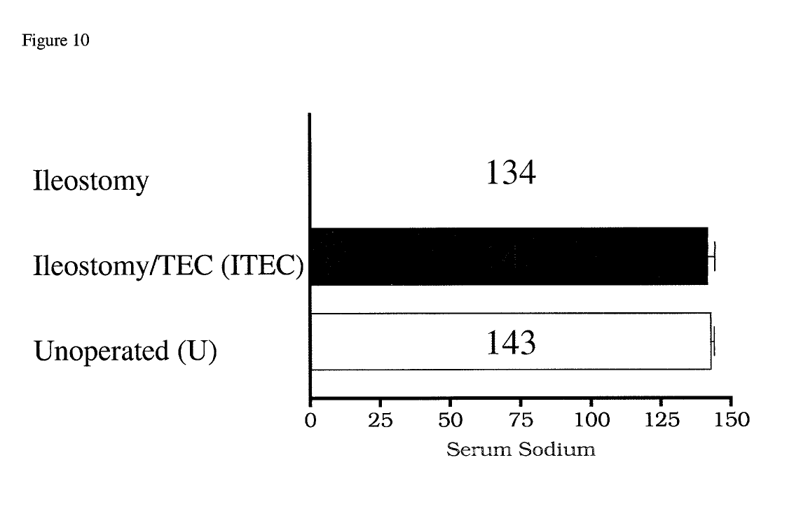

Blood Urea Nitrogen (BUN) levels were significantly higher in IL animals (24.25 mg/dl+/−1.708) than in ITEC animals (21+/−4.183) or normal control animals (18.6+/−0.8944, p=0.035). The difference in BUN between ITEC and normal control animals was not statistically significant. Similarly, sodium (FIG. 10) was lower in IL animals (134 mmol/L+/−2.7) than both ITEC animals (141.8+/−2.6) and unoperated control animals (143+/−1.2, p=0.0050). Again the difference between ITEC and normal control animals was not significant.

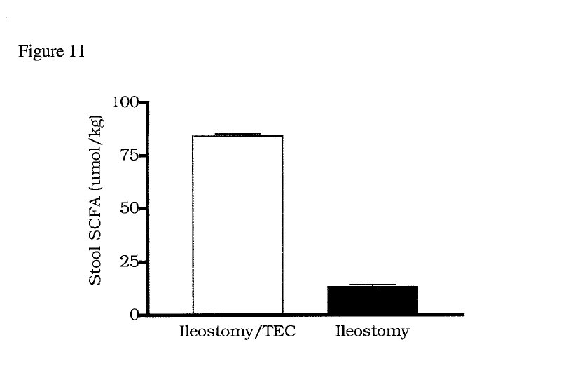

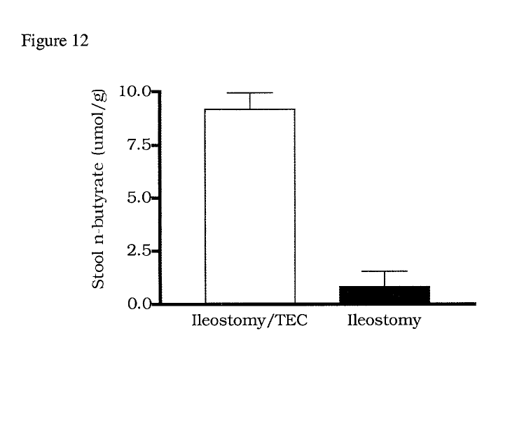

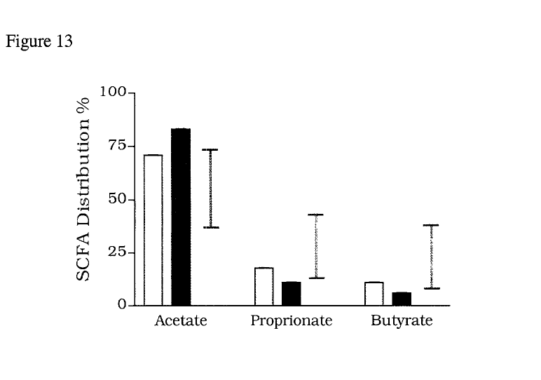

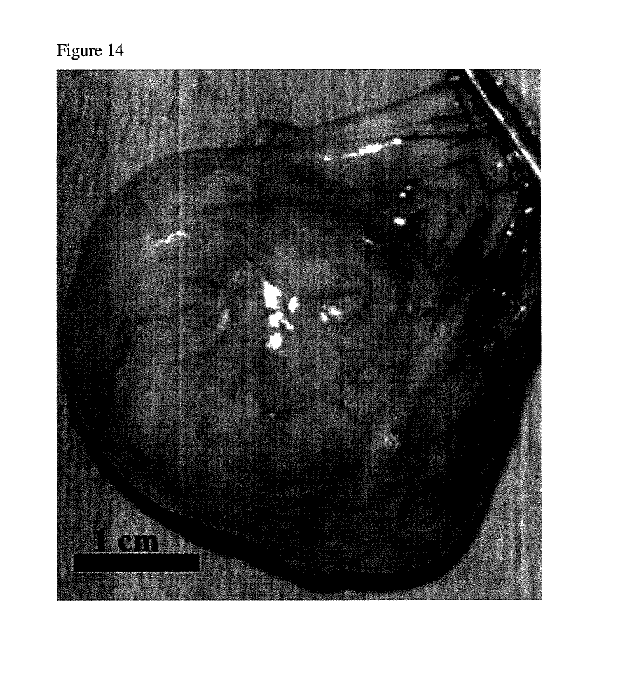

Stool short chain fatty acids (FIG. 11) were higher in ITEC animals (84.2 μmole/kg+/−0.5) than IL animals (13.5+/−0.5), with ITEC animals also having higher levels of n-butyrate (9.2 μmole/g+/−0.4) than IL (0.8+/−0.4) animals (FIG. 12). The distribution of short chain fatty acids (FIG. 13) was skewed in IL animals (83% Acetate, 11% Proprionate, 6% n-butyrate) and within normal limits in ITEC animals (71%, 18%, 11%). Fecal lactoferrin was not identified in either group.

Stool bacteriology was similar, with E. coli, Bifidobacterium, alpha hemolytic strep, gamma hemolytic strep, P. mirabilis, beta Strep (Not Group A or B) and Haemolytic E. coli as the major colonizers.

Taken together, these results demonstrate that tissue-engineered colon can generate functional living tissue, recapitulating the function of native colon.

### Example 2

### Tissue-Engineered Large Intestine Derived from Neonatal, Adult and Engineered Tissue Resembles Native Colon with Appropriate In Vitro Physiology and Architecture

Organoid units, mesenchymal cell cores surrounded by a polarized epithelia derived from full thickness sigmoid colon dissection from neonatal Lewis rats, adult rats, and tissue-engineered colon (TEC), were implanted on a polymer scaffold into the omentum of syngenic hosts. TEC was either anastomosed at four weeks or excised for Üssing chamber studies or histology, immunohistochemistry, and TUNEL assays. All animals generated TEC without regard to tissue source (e.g. neonatal, adult, or engineered tissue). Tissue-engineered colon can be successfully produced with fidelity to native architecture and in vitro function from neonatal syngenic tissue, adult tissue, and tissue-engineered colon itself.

Organoid Unit Preparation

Colon organoid units were produced by dissecting the sigmoid colon without mesentery from four-day-old Lewis rat pups (n=40) and applying the methods described in Example 1. Organoid units were reconstituted in high glucose DMEM with 10% iFBS, counted by hemocytometer, and loaded with approximately 100,000 units per polymer at 4° C., maintained at that temperature until implantation, which occurred in under 1.5 hours.

Polymer Scaffolds

Polymer scaffolds were constructed as described in Example 1.

Implantation of Tissue-Engineered Colon into Rat Omentum

Under pentobarbital anesthesia, thirty 150 g Lewis rats were implanted with tissue-engineered colon (TEC). Implantation was achieved as described in Example 1. Four weeks later, TEC was either harvested for Üssing chamber experiments (n=10), histology and immunohistochemistry (n=8) or anastomosed to either small intestine (n=5) or large intestine (n=5) in a side-to-side fashion with 6-0 Prolene. The remaining two animals were harvested at 4 weeks and organoid units were generated from their TEC and re-implanted into eight syngenic hosts according to the methods described in Example 1. Similarly, adult colon was harvested from two 400 g six-month old Lewis rats according to the methods described in Example 1 and implanted in eight hosts.

Animals

All animals were given a normal diet. They were allowed free access to lab chow and water, consuming approximately equal amounts after postoperative day 1. Pouch size was measured at time of anastomosis and at harvest (day 31 for all anastomosed animals).

Polymer Variation

In order to establish the optimal polymer configuration for the generation of TEC prior to the studies above, organoid units were obtained as described and seeded 100,000 units per 1.5 cm². Each construct was implanted in three 150 g Lewis rats and the resulting cysts were measured, studied with Hematoxylin and Eosin (H&E), Periodic Acid-Schiff (PAS), and trichrome. The implanted constructs were the following (Table 1): each group was implanted with or without a 5% coating of Poly (L-Lactic acid) and with either 0.5 mm thick nonwoven polyglycolic acid (PGA) tubes (OD 0.5 mm, length 6 cm) or 1 mm thick nonwoven PGA tubes (OD 1 cm, lengths 1, 3, 5, and 7 cm). Five additional groups having the same parameters were woven into the mesentery rather than the omentum, 2 mm thick nonwoven PGA (1 cm×1 cm square) and 1 mm thick nonwoven PGA (1 cm×1 cm square).

TABLE 1

| | | 5% Poly (L-Lactic acid) | |
|---|---|---|---|
| | Implantation | Coating | |
| Construct | Site | With | Without |
| 0.5 mm nonwoven PGA | Omentum | + | |
| (OD 0.5 mm, length 6 cm) | | | + |
| 1 mm nonwoven PGA | Omentum | + | |
| (OD 1 cm, length 1 cm) | | | + |
| 1 mm nonwoven PGA | Omentum | + | |
| (OD 1 cm, length 3 cm) | | | + |
| 1 mm nonwoven PGA | Omentum | + | |
| (OD 1 cm, length 5 cm) | | | + |
| 1 mm nonwoven PGA | Omentum | + | |
| (OD 1 cm, length 7 cm) | | | + |
| 0.5 mm nonwoven PGA | Mesentery | + | |
| (OD 0.5 mm, length 6 cm) | | | + |
| 1 mm nonwoven PGA | Mesentery | + | |
| (OD 1 cm, length 1 cm) | | | + |
| 1 mm nonwoven PGA | Mesentery | + | |
| (OD 1 cm, length 3 cm) | | | + |
| 1 mm nonwoven PGA | Mesentery | + | |
| (OD 1 cm, length 5 cm) | | | + |
| 1 mm nonwoven PGA | Mesentery | + | |
| (OD 1 cm, length 7 cm) | | | + |
| 2 mm nonwoven PGA | Mesentery | + | |
| (1 cm × 1 cm square) | | | + |
| 1 mm nonwoven PGA | Mesentery | + | |
| (1 cm × 1 cm square) | | | + |

Üssing Chamber Experiments

Non-fasted rats were anaesthetized and a midline laparotomy was performed; the anastomosed colonic cyst was gently freed from any adhesions and excised off its anastomosis with the native bowel. It was quickly rinsed free of luminal content in cold modified Ringer's solution. Similarly, a 5 cm length of native colon 5 cm distal to the cecum was excised, opened along its mesenteric border, rinsed and placed in cold Ringers' to serve as a control for the anastomosed colonic cyst in the Üssing chamber.

The cyst and native colon were mounted on a Lucite Üssing chamber block (World Precision Instruments, Sarasoto, Fla.) with an exposed area of 0.64 cm. The native colon was stripped of muscularis under a stereoscopic microscope whilst the cyst was partially stripped. The tissue was allowed to equilibrate in 10 ml of modified Ringers' solution containing 140 mM Na⁺, 5.4 mM K⁺, 1.3 mM Ca²⁺, 1.2 mM Mg²⁺, 2.4 mM HPO₄²⁻, 124 mM Cl⁻, 0.6 mM H₂PO₄⁻, 21 mM HCO₃⁻, 5 mM HEPES and 10 mM fructose in both the mucosal and serosal sides at pH 7.4±0.01. The tissue was gassed with a carboxygen ratio of 5/95% and maintained at 37° C. with water-jacketed reservoirs. Transmural potential difference (PD) was measured using calomel electrodes connected to the bathing solution with Ringer-Agar (3%) bridges. Tissues were continuously short-circuited with an automatic voltage clamp (model EVC-4000, World Precision Instruments) except during a 5-10 second interval every 5-10 minutes when the open-circuit PD (mV) was measured. A transmucosal potential difference PD (mV) was read and tissue resistance R (Ohm·cm²) was calculated from Ohm's law (V=IR), using Isc (μAmp/cm²) the short circuit current. Tissues from the same animal were paired. After a 30-45 minute equilibration period with modified Ringer's solution, both the mucosal and serosal sides of the tissue were exposed to a final chamber concentration of 30 mM 3-OMG. Changes in δIsc within 20-40 minutes was noted. Tissue viability was checked with a serosal chamber concentration of 5 mM theophylline. The maximum change in δIsc was also noted.

Results

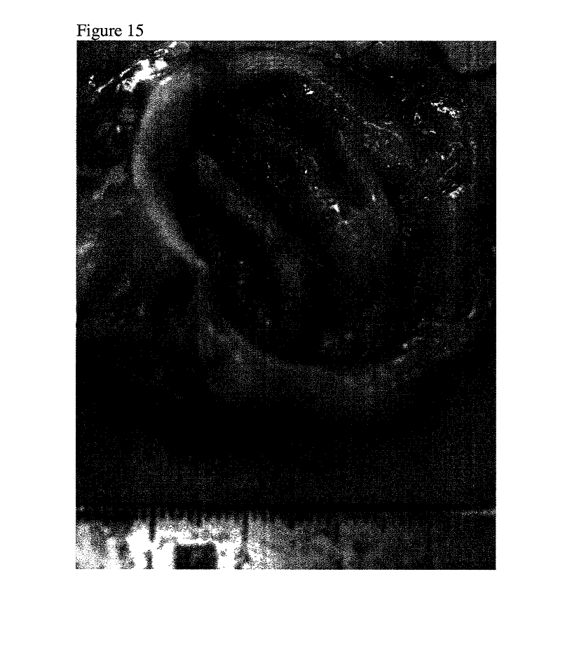

All animals survived implantation and all animals generated tissue-engineered colon, including those generating TEC from TEC itself and those implanted with TEC derived from adult tissue. The average size of the TEC was 4 cm×5 cm×4 cm with no TEC measuring less 30 than 3 cm×3 cm×2 cm. FIG. 14 shows a representative TEC cyst after four weeks in the omentum, and the same cyst, lumen exposed, in FIG. 15. The largest neointestine was 6 cm×8 cm×7.5 cm. Results on Üssing chamber studies and histology did not change with regard to the source of the organoid units, and four-week size was equally distributed between host sources. Therefore, the data described below is reported for all groups studied.

Tissue-Engineered Colon Formation

The initial studies of polymers resulted in two findings; the implanted TEC construct must stent the omentum open, rather than let it collapse around the polymer, and the omentum is the preferred TEC implantation site (Table 2).

TABLE 2

| | With | Without | With | Without |
|---|---|---|---|---|
| | Poly (L- | Poly (L- | Poly (L- | Poly (L- |
| | Lactic | Lactic | Lactic | Lactic |
| | acid), | acid), | acid), | acid), |
| Construct | Omentum | Omentum | Mesentery | Mesentery |
| Tube: 2 mm/0.5 cm/1 cm | + | − | − | − |
| Tube: 0.5 mm/0.5 cm/ | + | − | − | − |
| 6 cm | | | | |
| Tube: 1 mm/1 cm/1 cm | + | − | − | − |
| Tube: 1 mm/1 cm/3 cm | + | − | − | − |
| Tube: 1 mm/1 cm/5 cm | + | − | − | − |
| Tube: 1 mm/1 cm/7 cm | + | − | − | − |
| Square: 2 mm/1 cm/1 cm | − | − | − | − |
| Square: 1 mm/1 cm/1 cm | − | − | − | − |

Polymers are listed by form (tube or square)/thickness of nonwoven PGA, outside diameter (OD)/length for tubes and form/x/y for squares. Formation of TEC cysts 3 cm×3 cm×2 cm in all hosts was graded “+” and formation of multiple discontinuous cysts or a cyst smaller than the standard was graded “−”. TEC size was not statistically significantly different when produced.

All animals formed TEC. However, constructs that were not tubular, coated with Poly (L-Lactic acid), and implanted in the omentum exhibited a potential to form structures comprising many small cysts that were not always contiguous along the path of the polymer. In contrast, any tubular polymer constructed of polyglycolic acid (PGA)/Poly (L-Lactic acid) and implanted in the omentum generated a TEC cyst as described above. Increased surface area of the construct did not increase the resulting size of the TEC generated, and there was no statistically significant difference in TEC size once generated. Implantation in the mesentery always resulted in discontinuous multiple small cysts of TEC whether the construct was coated or not.

Tissue-Engineered Colon Histology and Immunohistochemistry

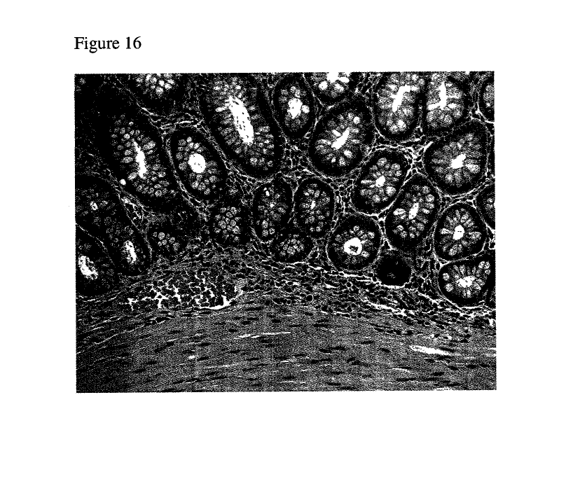

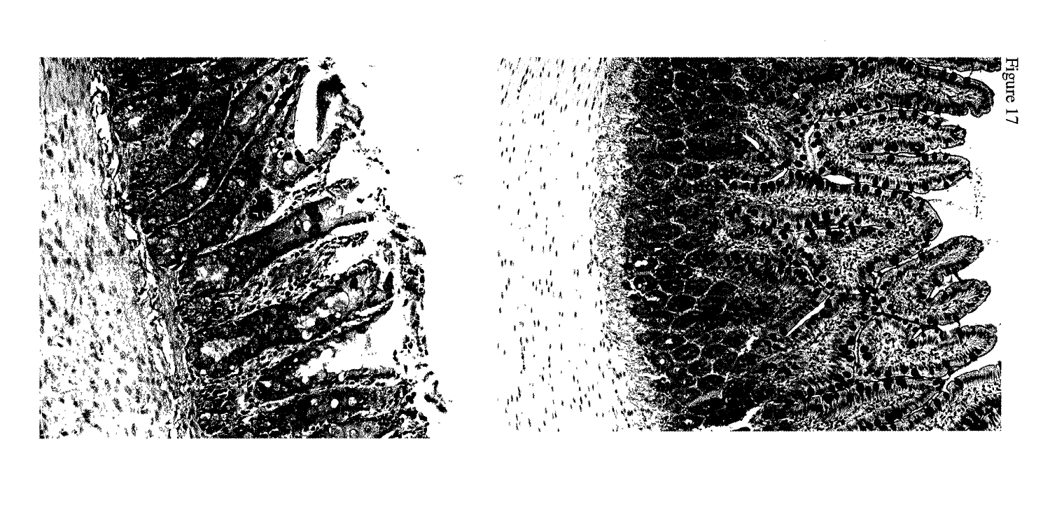

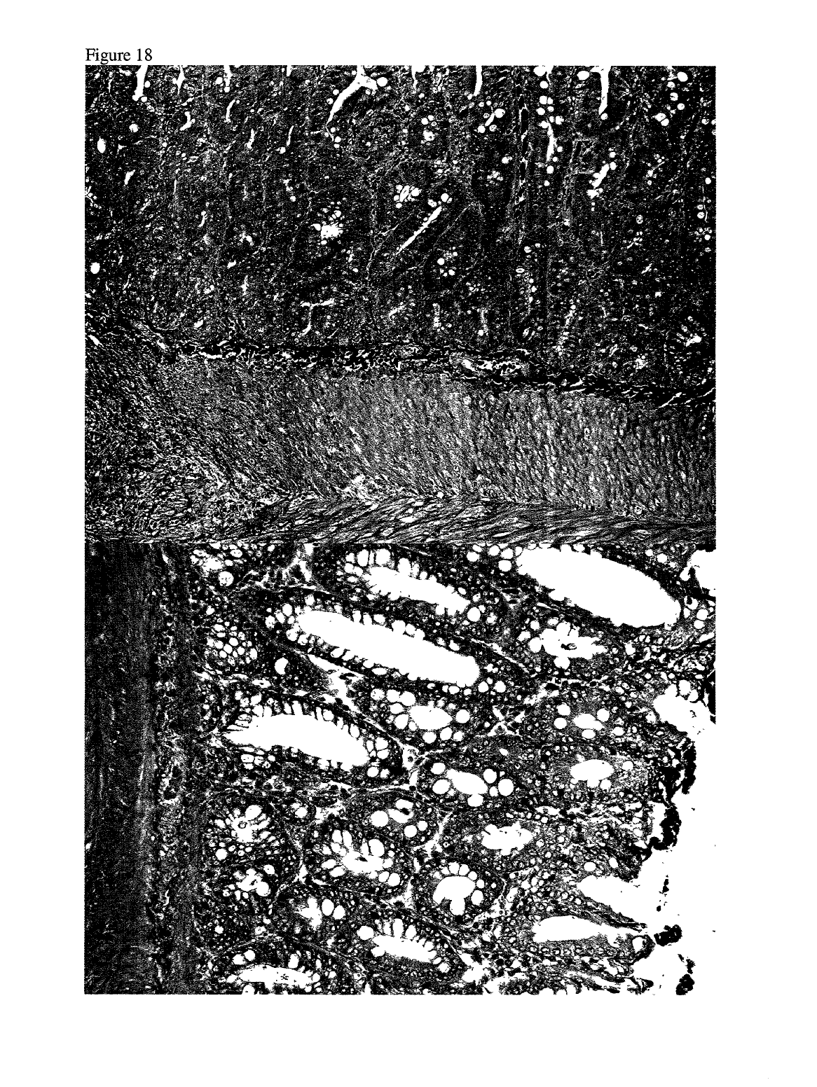

Formalin-fixed tissue was sectioned and stained with H&E, PAS, and Trichrome staining to observe tissue architecture and for evidence of inflammation according to St. Mark's criteria as described in Example 1. Hematoxylin and Eosin (H&E) staining of TEC revealed an uninterrupted uniform intestinal epithelium facing the lumen of the cysts with an absence of villi or Paneth cells, long crypts of Lieberkuhn with numerous goblet cells, and a loose lamina propria bearing lymphoid cells. There was an outer longitudinal layer of smooth muscle, normal vascularization, present ganglion cells, and no evidence of inflammation (FIG. 16). Periodic Acid-Schiff (PAS) staining (FIG. 17) showed a normal distribution of goblet cells. Trichrome staining revealed a normal collagen rich blue submucosa (FIG. 18).

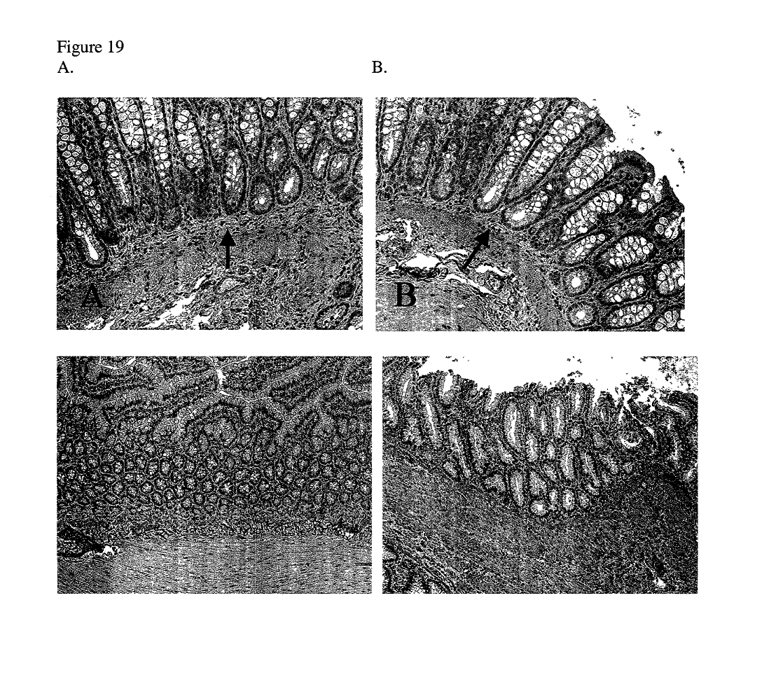

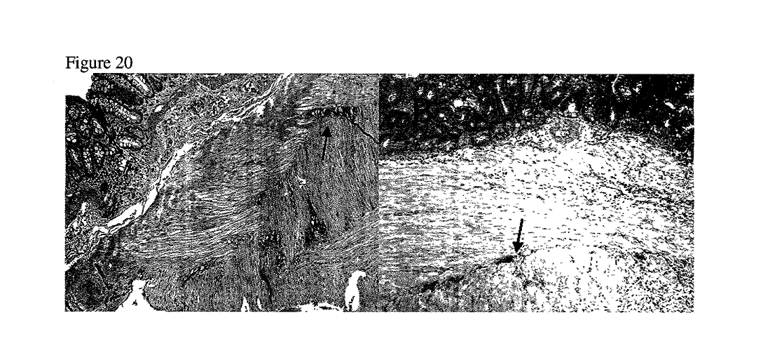

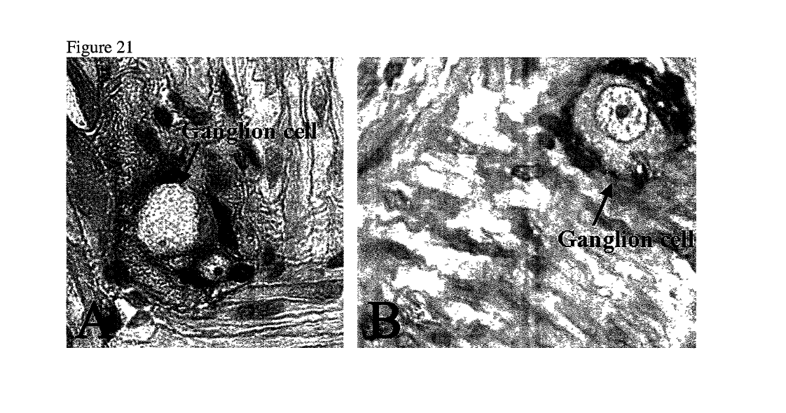

Immunohistochemistry was performed using the DAKO Envision kit for alpha anti-smooth muscle actin (Sigma) and the S100 protein (DAKO). Staining for smooth muscle actin revealed normal colon architecture with smooth muscle staining in the muscularis propria similar to that of native colon (FIG. 19). S100 staining revealed dense staining in the areas of Auerbach's and Meissner's plexi with less density in the TEC but appropriate positioning (FIG. 20). In the midst of the S 100 positive cells, large lucent ganglion cells were identified in both native and tissue-engineered colon (FIG. 21).

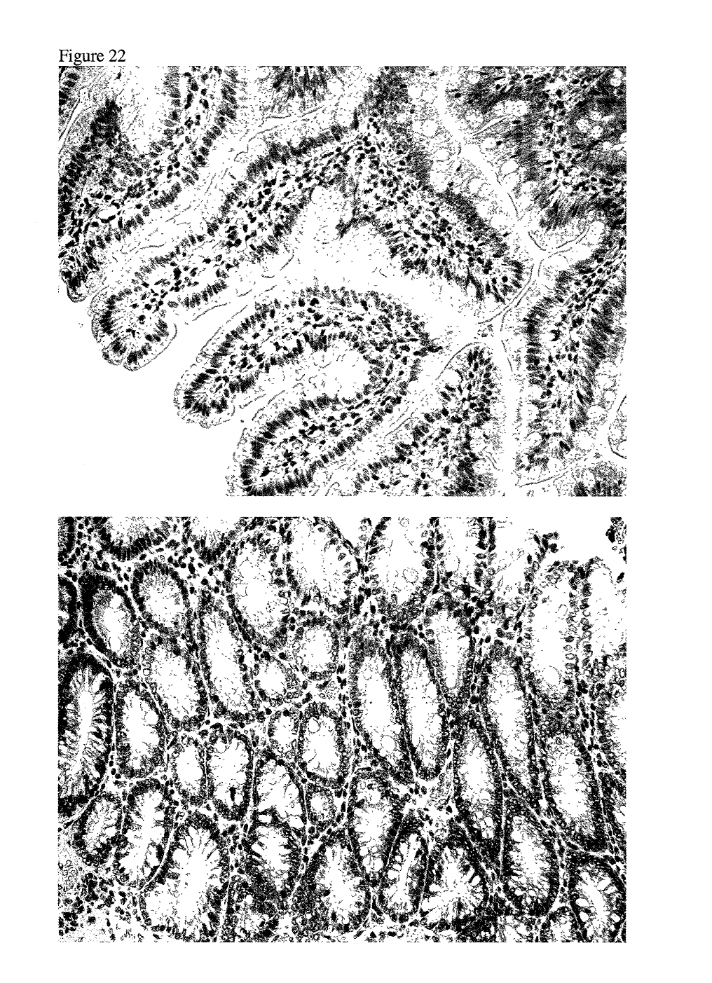

The TUNEL (Tdt-mediated dUTP Nick End Labeling) assay (FIG. 22) was performed using the ApopTag kit (Intergen). It identified identical numbers of positive cells in native colon and TEC, 4 per high power field (hpf).

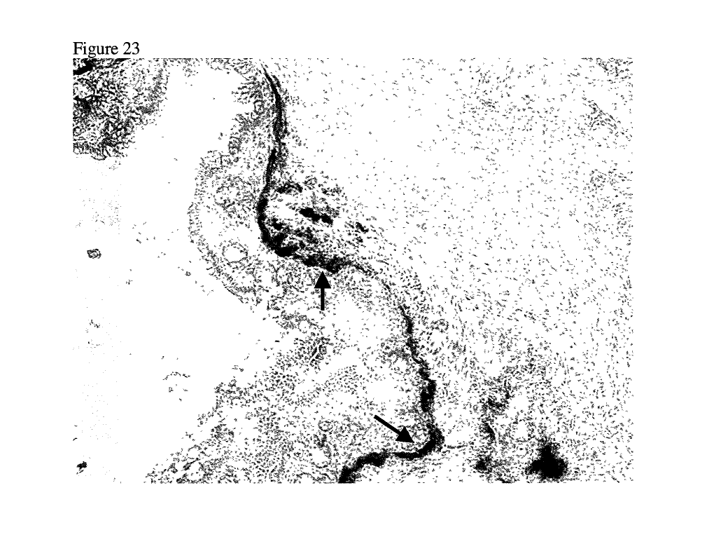

Frozen sections were collected and stained with Kamovsky's fluid for anti-acetylcholinesterase. Anti-acetylcholinesterase staining was visible in a normal contiguous distribution in the lamina propria without tufting and with an absence of staining in the muscularis mucosa, as seen in normal colon (FIG. 23).

Transmission Electron Microscopy

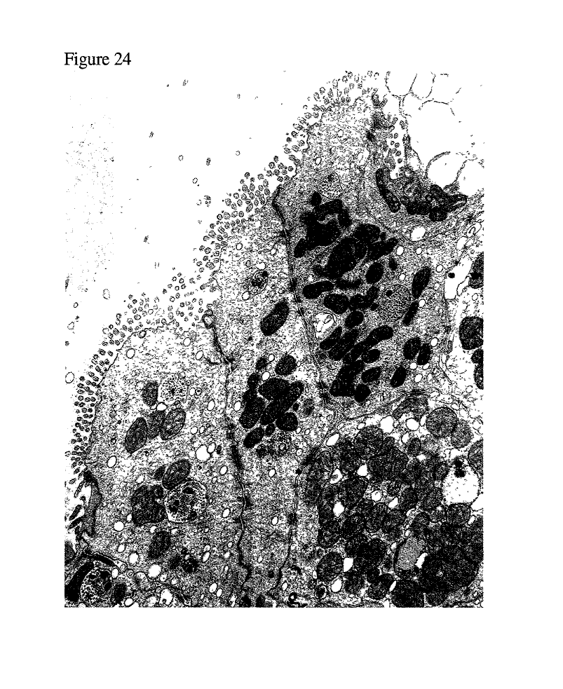

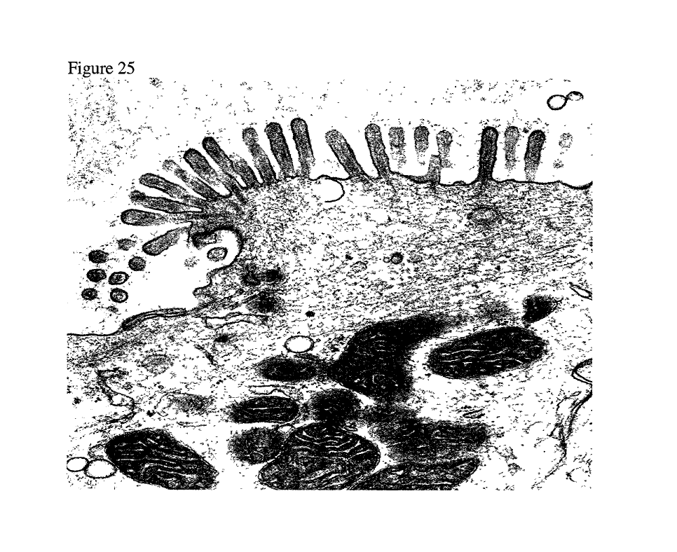

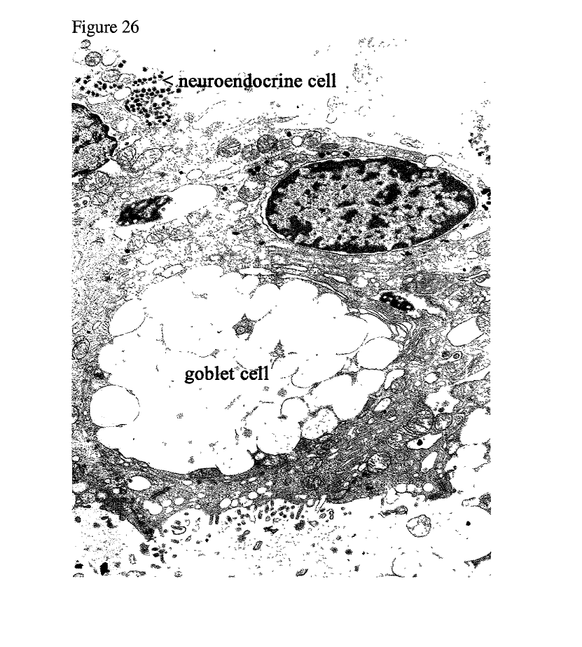

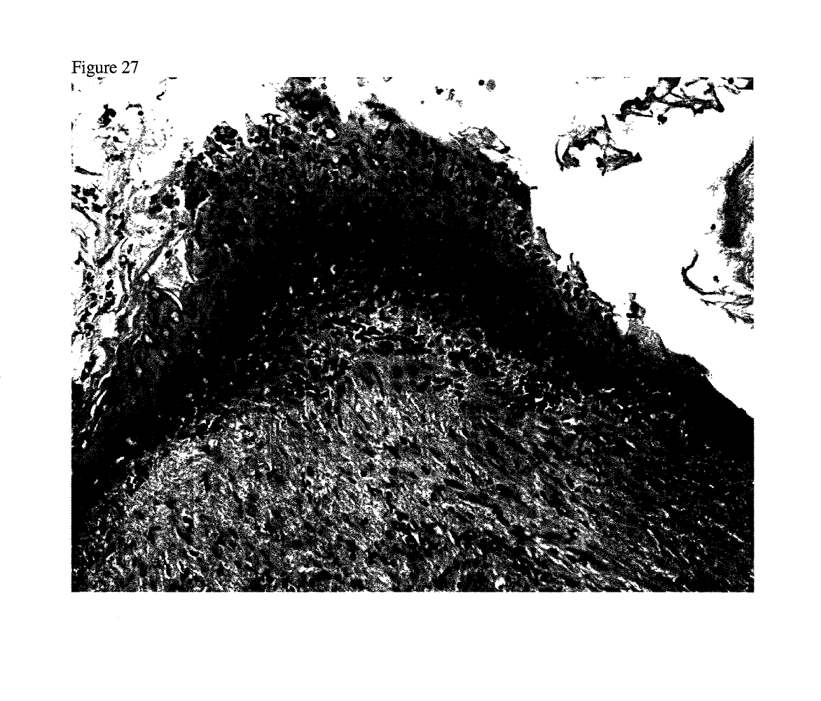

Transmission Electron Microscopy (TEM) showed normal microarchitecture, with tight junctions, abundant mitochondria, desmosomes with keratin filaments (FIG. 24), apical microvilli (FIG. 25), neuroendocrine cells, and goblet cells (FIG. 26).

Architecture of Tissue-Engineered Colon

The same observations except for TEM, which was not repeated, were made of TEC after anastomosis, and whether anastomosed to small intestine or large intestine, the architecture of the TEC was preserved. The afferent loop of small intestine carried liquid contents into the cyst, which contained homogenous solid contents that were expressed via the efferent loop of the small intestine. At harvest, the stool contents above the TEC whether anastomosed to small intestine or large intestine were grossly more liquid than the contents that exited the anastomosed area, reflecting overall fluid absorption by the TEC segment.

Electrophysiological Parameters

The electrophysiological parameters of native colon and anastomosed tissue-engineered cyst were evaluated in the Üssing chamber (Summarized in Table 3).

TABLE 3

| Üssing Chamber Observation | Correlation |
|---|---|
| Spontaneous short-circuit current | Active ion transport |
| No short-circuit current response | Absence of epithelial SGLT1 expres- |
| to 3-O-methylglucose addition | sion, consistent with the presence |
| | of mature colonocytes |
| Positive short-circuit current | Intact secretagogue-induced chloride |
| response to theophylline | secretion and tissue viability. |

The following values were obtained at equilibrium Potential Difference (PD) (mV), Isc (μAmp/cm2) and R (Ω·cm2) for native colon and anastomosed cyst 4.85±1.22 mV, 47.0±6.38 μAmp/cm2, 63.0±8.011 Ω·cm2 vs. −1.60±0.44 mV, 13.25±3.20 μAmp/cm2 and 82.95±19.70 Ω·cm2 respectively (n=4). There was no change in δIsc to 30 mM 3-OMG from both tissues (n=4). The maximum change in δIsc after theophylline was 72.5±13.96 vs. 11.67±3.96 μAmp/cm2 for native colon and anastomosed cyst respectively (n=3). The percent δIsc to theophylline was 274.87±49.72 for native colon and 217.59±48.30 anastomosed cyst.

In summary, TEC architecture was identical to native architecture with muscularis propria staining for actin, acetylcholinesterase detected a linear distribution in the lamina propria, S100 positive cells, ganglion cells, and a TUNEL assay similar to native colon. Üssing chamber data indicated in vitro function consistent with mature colonocytes, and a positive short circuit current response to theophylline indicating intact ion transfer. Transmission Electron Microscopy (TEM) showed normal microarchitecture.

Thus, tissue-engineered colon recapitulated native colon in form and physiological function, irrespective of tissue source (e.g., neonatal, adult or engineered tissue).

### Example 3

### Tissue-Engineered Esophageal Replacement in a Rat Model

Organoid units, derived from the esophagus of Lewis rats, were implanted on to a polymer scaffold into the omentum of syngenic hosts. Tissue-engineered esophagus (TEE) was either anastomosed at four weeks or excised for histology and immunohistochemistry. All animals generated TEE. Tissue-engineered esophagus can be successfully produced with fidelity to recapitulate native architecture and in vitro function, and can replace native esophagus in vivo.

Organoid Unit Preparation

Esophageal organoid units were produced by harvesting a portion of the esophagus from three-day-old Lewis rats (n=30), although the organoid units can be generated from adult (FIG. 27) as well as neonatal rats, so the donor age is not critical. Esophageal organoid units were washed, dissected, enzymatically digested and centrifuged with purification to multicellular units as described in Example 1.

Polymer Scaffolds

Polymer scaffolds were constructed of 1 cm long polyglycolic acid polymer tubes, sealed with 5% Poly (L-Lactic acid), and coated with collagen.

Implantation of Tissue-Engineered Esophagus into Rat Omentum

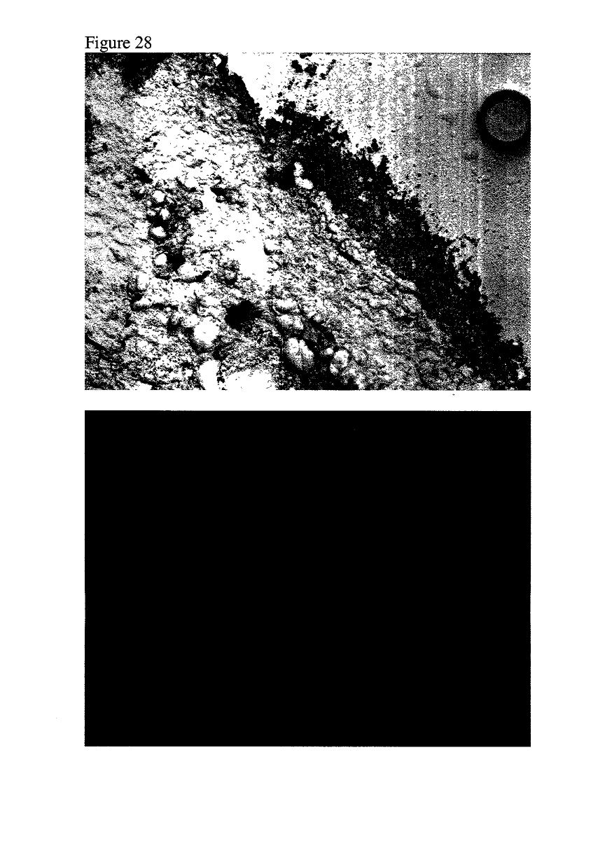

Seven 150 g Lewis rats were implanted with tissue-engineered esophagus (TEE). Implantation was achieved as described in Example 1. The implantation was also performed with esophageal organoid units that had been labeled with GFP for one hour at 37° C. to distinguish and verify engineered tissue from host tissue (FIG. 28). Confirmation of GFP production was obtained by simultaneous in vitro culture of the organoid units. Four weeks later, TEE was examined through a midline laparotomy and either harvested for histology (n=5) or anastomosed after resection of the native abdominal esophagus (n=2). Resection was accomplished by removing a section of the native abdominal esophagus 3.5 cm×1 cm×1 cm. A connecting strand less than 0.5 mm was maintained in order to tether the two ends of esophagus during anastomosis. Tissue-engineered esophagus was anastomosed in an end-to-end fashion above the lower esophageal sphincter (LES).

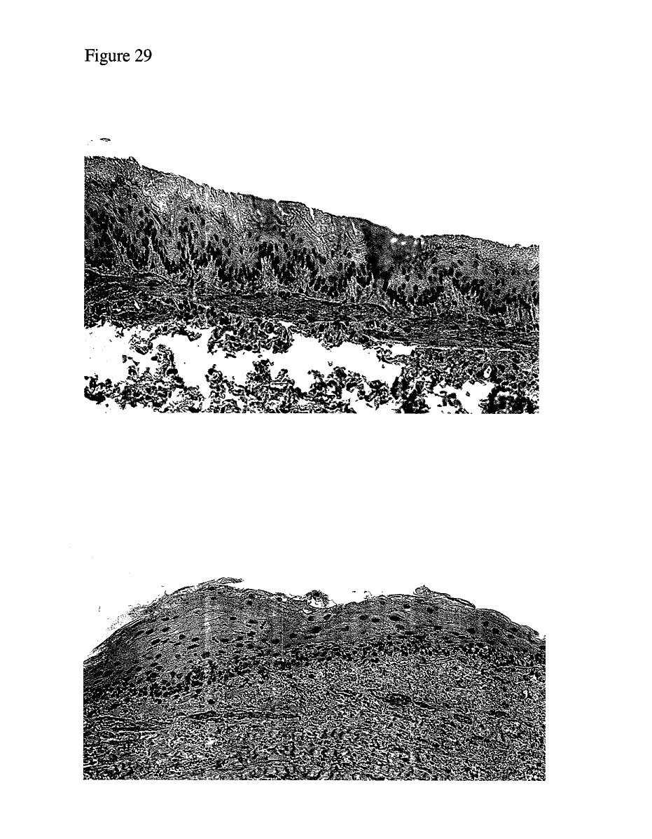

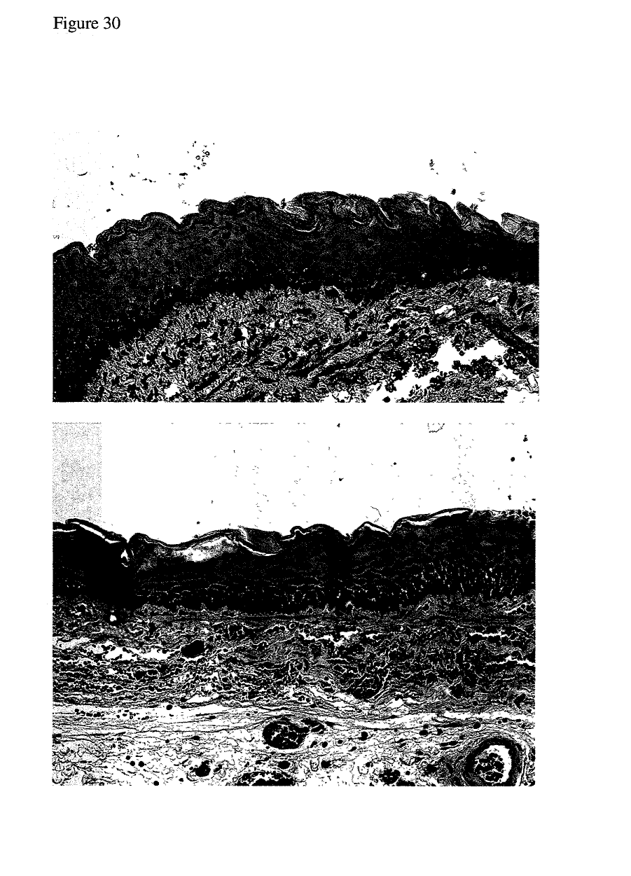

Results All seven rats grew tissue-engineered esophagus measuring approximately 3.5 cm×3 cm×2 cm. H&E and immunohistochemistry for smooth muscle actin was performed (FIG. 29). The omental cysts were an exact recapitulation of rat abdominal esophagus anatomy, containing the normal orientation of squamous epithelium (FIG. 30) with all relevant layers, a muscularis mucosa stained positive for smooth muscle actin, submucosa, and muscularis propria. Esophageal replacement with acellular control organoid units resulted in animal death by 24 hours.

Weights were measured QOD (n=2) for 42 days and compared to esophageal replacement by an acellular omental construct (n=2). Replacement with tissue-engineered esophagus resulted in an initial weight loss (minimum weight 87.5% and 87.67% preoperative weight on days 5 and 7) followed by a linear weight gain; animals were 108% and 110% preoperative weight on day 42.

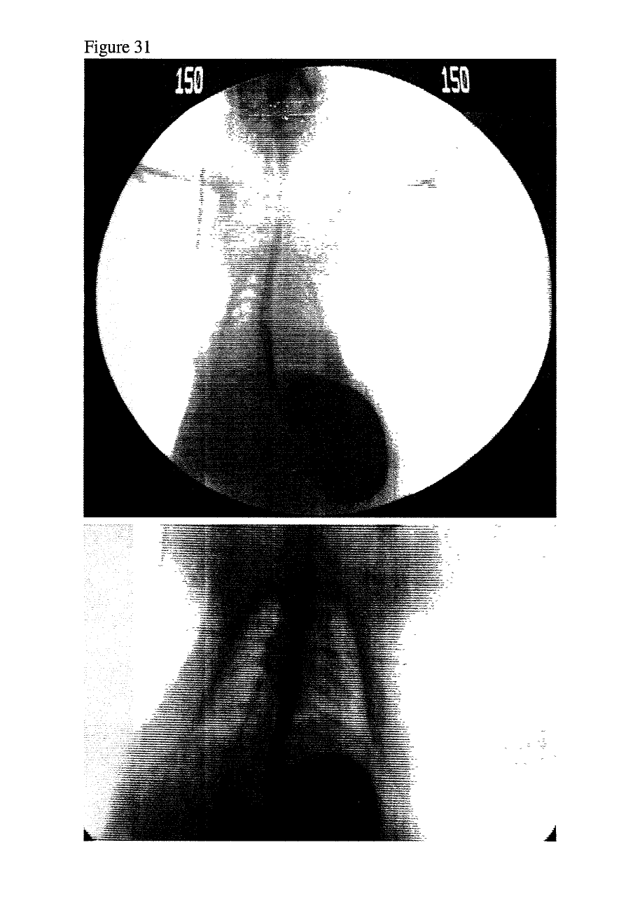

Upper gastrointestinal fluoroscopy was performed at 42 days (n=2) and compared to unoperated control animals. Fluoroscopy revealed a smooth lumen with no evidence of obstruction, a wider anastomosed section consistent with the dimensions implanted, and passive contraction of the engineered segment after contrast injection (FIG. 31).

Histology revealed appropriate continuous abdominal esophageal architecture including epithelial and muscular elements. Tissue-engineered esophagus can be successfully produced with the appropriate architecture and to replace native esophagus in vivo.

### Example 4

### Tissue-Engineered Esophagus Functions as Interposition Graft

Organoid units, derived from the esophagus of Lewis rats, were implanted on to a polymer scaffold into the omentum of syngenic hosts. Tissue-engineered esophagus (TEE) was harvested for histology or double end to end anastomosed after resection of distal native esophagus. All animals generated TEE. Tissue-engineered esophagus had the appropriate abdominal esophageal architecture and can successfully replace native esophagus in vivo.

Organoid Unit Preparation

Esophageal organoid units were produced by harvesting the lower esophagus from four-day-old Lewis rats (n=40), although the organoid units can be generated from adult as well as neonatal rats, so the donor age is not critical. Esophageal organoid units were digested, centrifuged, and purified to multicellular units as described in Example 1.

Polymer Scaffolds

Polymer scaffolds were constructed of 1 cm long polyglycolic acid polymer tubes.

Implantation of Tissue-Engineered Esophagus into Rat Omentum

Ten adult Lewis rats were implanted with tissue-engineered esophagus (TEE). Implantation was achieved as described in Example 1. After four weeks, TEE grew and was harvested for histology (n=5) or double end to end anastomosed after resection of 5 cm of distal native esophagus including the lower esophageal sphincter (LES).

Results

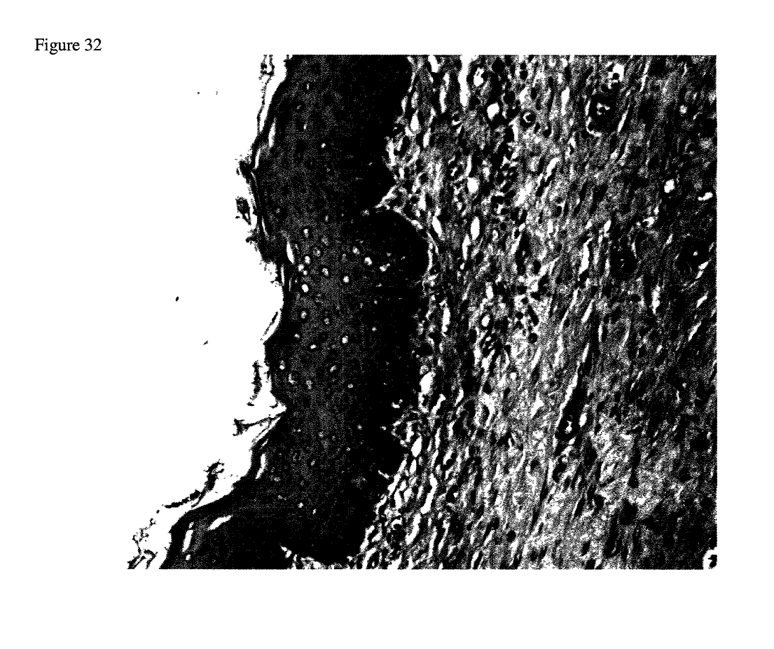

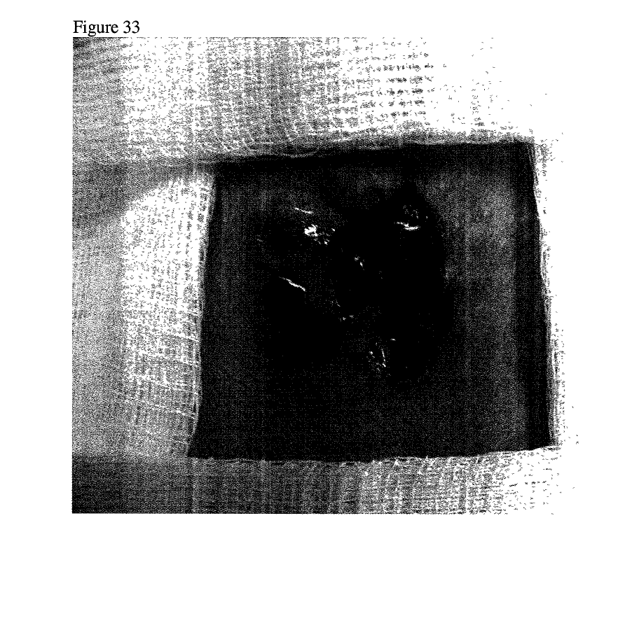

All ten rats grew tissue-engineered esophagus cylinders measuring approximately 4 cm×3 cm×2 cm. H&E (FIG. 32) and actin immunohistochemistry were performed. TEE was equivalent to rat abdominal esophagus, containing squamous epithelium with all layers, muscularis mucosa staining for actin, submucosa, and muscularis propria. Esophageal replacement with acellular controls resulted in animal death by 24 hours.

Weights were measured QOD for 20 days and compared to esophageal replacement by an acellular construct and TEE onlay patch. TEE replacement resulted in an initial weight loss (minimum weight 78% preoperative weight vs. 86% for onlay patch at day 7) followed by a linear weight gain. Animals were 94% preoperative weight on Day 20.

Upper gastrointestinal fluoroscopy was compared to unoperated controls. Fluoroscopy on Day 20 showed a smooth lumen with no evidence of obstruction, a wider anastomosed section consistent with implant dimensions, and passive contraction after contrast injection.

Tissue-engineered esophagus has the appropriate abdominal esophageal architecture including epithelial and muscular elements. It successfully replaced native esophagus in vivo.

### Example 5

### The Generation of Splenic Mass Using Tissue Engineering

Organoid units, derived from the spleen of Lewis rats, was implanted on to a polymer scaffold into the omentum of syngenic hosts. Tissue-engineered spleen (TES) was harvested and analyzed by H&E, immunohistochemistry, dry weight, DNA assays. All animals generated TES with native architecture.

Organoid Unit Preparation

Spleen organoid units were produced by dissecting spleens from four-day-old Lewis rats (n=20), although the organoid units can be generated from adult as well as neonatal rats, so the donor age is not critical. Spleen organoid units were digested and triturated into uniform multicellular splenic units as described in Example 1.

Implantation of Tissue-Engineered Spleen into Rat Omentum

Spleen organoid units were coated on a reticular framework composed of highly porous polyglycolic acid. TES constructs were implanted into the omentum of male Lewis rats with (TES+, n=8) or without splenectomy (TES−, n=20). Implantation was achieved as described in Example 1. TES constructs were harvested at weeks 2, 3, and 8.

Statistics

Statistics were performed by Mann-Whitney and ANOVA, reported +/−SEM.

Results

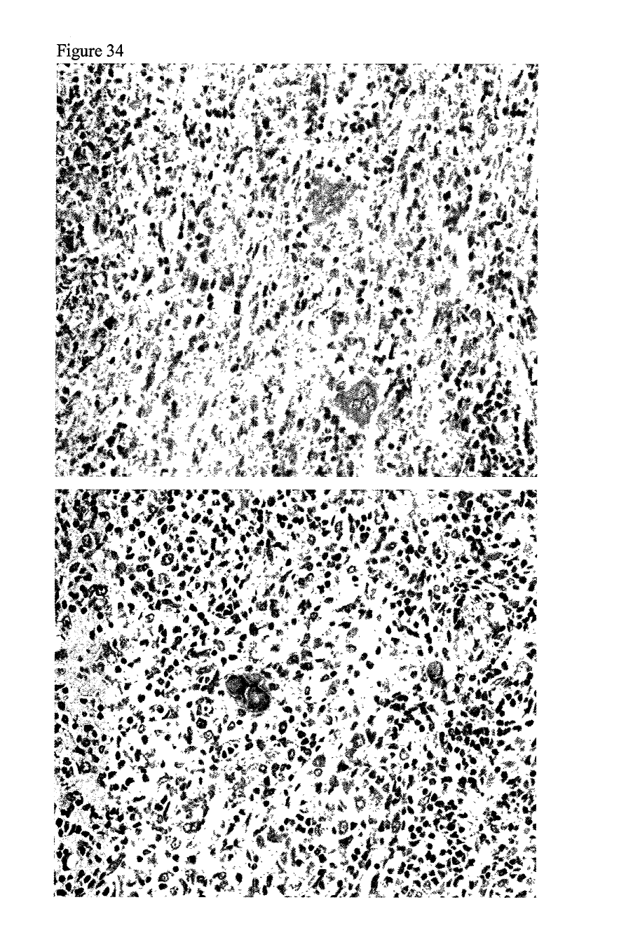

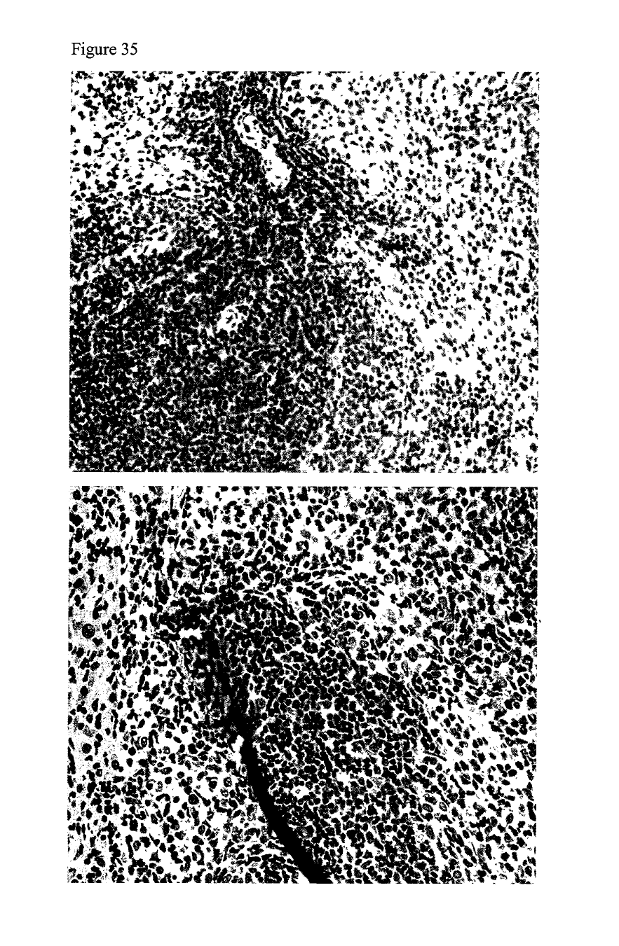

At two months all animals generated visible deep purple TES (FIG. 33). Histology revealed organized spleen parenchyma with white pulp lacking germinal centers organized around arteries that stain for von Willebrand factor (vWF) and red pulp with venous sinuses (FIG. 34). CD3 immunohistochemistry stained the interfollicular zone (FIG. 35). In early TES, there was developing splenic architecture with no evidence of necrosis. TES+ dry weight was 66.4+/−4.4 mg per specimen. TES− dry weight was 70.8+/−7 mg. Both were significantly greater than polymer with splenic units (38.26+/−0.09 mg, p<0.03). DNA content of TES− (7,836 ng DNA/mg dry weight), TES+ (9,456 ng DNA/mg dry weight), and native spleen (8,223 ng DNA/mg dry weight) were not significantly different.

The total splenic volume increased in TES animals without splenectomy. All animals generated TES with native architecture. These results indicate that this technique can also apply to the generation of other engineered organs or components of the immune system, including but not limited to the thymus and bone marrow.

### Example 6

### Tissue-Engineered Small Intestine

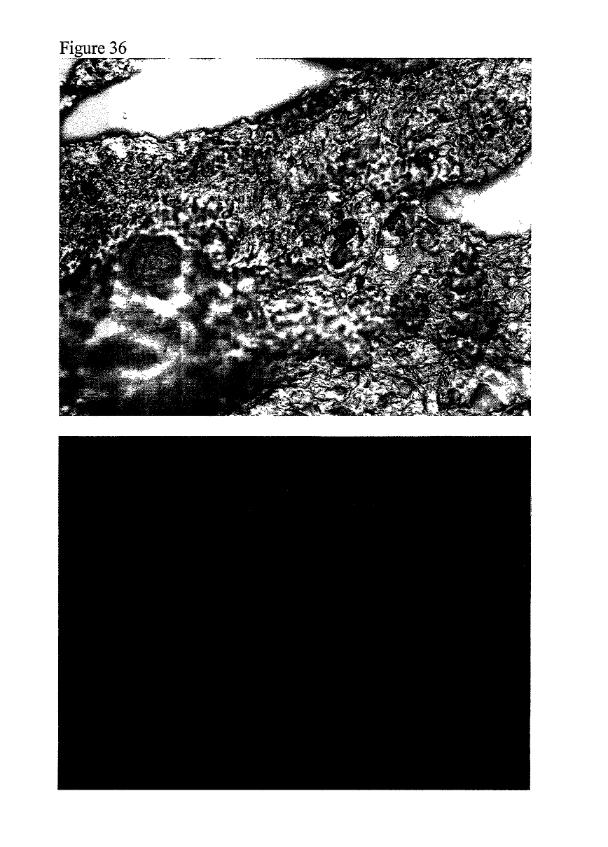

Neointestinal cysts were engineered by seeding biodegradable polymers with neonatal rat intestinal organoid units. The implantation was also performed with small intestine organoid units that had been labeled with GFP to distinguish and verify engineered tissue from host tissue (FIG. 36). Confirmation of GFP production was obtained by simultaneous in vitro culture of the organoid units. The cysts were matured and anastomosed to the native jejunum of syngenic adult recipients. Histology and surface area calculations were performed, as well as Northern blot and localization analysis of the Na+/glucose cotransporter (SGLT1). GLP-2 treatment augmented the absorptive surface area of engineered intestine was increased.

Results

Animals were treated with [Gly2]GLP-2 (twice daily, 1 μg/g body weight), a long-acting analogue of GLP-2, or vehicle alone (control) for 10 days. Rats were then sacrificed and tissues harvested for analysis. Histological parameters and surface area determinations were done using computer-based morphometry. GLP-2 administration resulted in a 40% expansion of absorptive surface area (data given as a mucosal amplification ratio, expressed as mean±S.D. in microns except for surface area; Table 4) and a two-fold increase in SGLT1 mRNA expression in the neointestine.

TABLE 4

| | | Villus | Crypt | |
|---|---|---|---|---|
| | Villus Height | Width | Width | Surface Area |
| Control | 261 ± 67 | 96 ± 17 | 52 ± 11 | 5.1 ± 0.8 |
| GLP-2 treated | 456 ± 23 | 114 ± 32 | 63 ± 6 | 7.3 ± 1.4 |
| | (p < 0.05) | | | (p < 0.05) |

SGLT1 mRNA expression was localized to enterocytes throughout the villi, and the SGLT1 protein was localized to the brush-border of enterocytes in the mid-villi and upper-villi of the neointestine. SGLT1 expression topography was unperturbed by GLP-2 administration.

Importantly, this approach yielded greater SGLT1 expression rather than neomucosa populated by less differentiated enterocytes. This is an effective strategy for increasing the absorptive surface area of engineered intestine.

### Example 7

### Tissue-Engineered Small Intestine Improves Recovery after Massive Small Bowel Resection

Organoid units, derived from the small intestine of Lewis rats, were implanted on to a polymer scaffold into the omentum of syngenic hosts. Tissue-engineered small intestine (TESI) was either anastomosed at four weeks or excised for histology and immunohistochemistry. All animals generated well-formed TESI that produced native small intestinal organization.

Implantation of Tissue-Engineered Small Intestine into Rat Omentum

Ten Lewis rats underwent implantation of TESI (TESI+, n=5). Implantation was achieved as described in Example 1. Four weeks later, an 85% bowel resection with end-to-end anastomosis was performed, leaving 5 cm of intestine distal to the ligament of Treitz and 5 cm of bowel proximal to the iliocecal valve. A side-to-side anastomosis of TESI to the upper 5 cm of bowel was performed (TESI+) or omitted (TESI−). Animals were sacrificed day 40.

Animals

Animals had access to water and lab chow after post-operative day 1 (POD1).

Results

Weights were measured QOD, analyzed by two-stage analysis or Mann-Whitney. All ten rats initially lost, then regained weight. The rate of weight loss was not statistically significantly different between TESI+ (−3.1% per day) and TESI− (−1.8% per day, p=0.068). On Day 40, TESI+ rats had regained a statistically significant greater percentage of their preoperative weight (98.533+/−3.519) than TESI− (76.8+/−2.354, p<0.003). The time to lowest weight was significantly shorter in TESI+ (7.7 days vs. 14.6 days, p=0.009). Maximum weight loss was significantly higher in TESI−, declining to 72.1% preoperative weight vs. 78.9% for TESI+, p=0.05. The rate of weight gain was higher in TESI+ (0.7% per day) than in TESI− (0.2% per day, p=0.004).

Bowel lengths and widths were measured at anastomosis and analyzed by Kruskal-Wallis. Bowel width significantly increased in both TESI+ (1.08 cm+/−0.05) and TESI− (0.893+/−0.02) compared to pre-resection width (0.5+/−0.01, p=0.001), and was not significantly different between the two groups. Final bowel length was not significantly different between TESI− (11.8 cm+/−0.4) and TESI+ (13+/−0.3). Both were greater than the initial bowel length (10.3+/−0.1, p=0.0005).

H&E and smooth muscle actin immunohistochemistry were performed. Histology revealed well-formed small intestine, positive for actin in the muscularis mucosa.

Blood was collected at sacrifice. Total protein, globulin, albumin, cholesterol, triglycerides, and metabolic profiles were not significantly different. Serum B12 was significantly higher in TESI+ (439.25+/−70 pg/ml) than TESI− (195.4+/−14, p=0.0159).

Anastomosis of tissue-engineered small intestine significantly improved the post-operative weight and B12 absorption of rats undergoing massive bowel resection, decreasing the time to lowest weight by a week, increasing the rate of weight gain, and diminishing the maximum weight loss. Small intestinal organization was conserved in TESI. Thus, all animals generated tissue-engineered small intestine with native small intestine structure.

### Example 8

### Angiogenesis in the Tissue-Engineered Intestine

Twenty-three engineered small intestinal cysts were harvested from Lewis rat recipients one to eight weeks after implantation. Architectural similarity to bowel obtained from juvenile rats (two-eight weeks old) was assessed with H&E stained sections. Over the eight week period, the small intestinal cysts increased in volume (0.5 cm³ at week one vs. 12.6 cm³ at week eight) and mass (1.3 g at week one vs. 9.7 g at week eight). The muscular and mucosal layers increased in thickness but the capillary density remained constant (mean±SEM: 82.95±4.81). The VEGF level was significantly higher in the juvenile rat bowel (147.6±23.9 pg/mg) compared to the engineered small intestinal cyst (42.3±3.4 pg/mg; p<0.001). Tissue bFGF levels were also higher in the juvenile rat bowel (315.0±65.48) compared to the engineered small intestinal cyst (162.3±15.09; p<0.05).

As the engineered intestine grew it regained some architectural features of normal bowel and the capillary density is maintained at a constant level. The mechanism driving angiogenesis differed between the engineered intestine and normal bowel. The neointestine increased in size and grew to resemble the native small bowel. The capillary density remained constant as the tissue expanded. The mucosal capillary density was similar in the neointestine, juvenile, adult intestine.

Animals

Adult (150-200 g) and three-day old Lewis rats, purchased from Charles River Laboratories (Wilmington, Mass.), were housed in accordance with the National Institutes of Health guidelines. The Harvard Medical Area Standing Committee on Animals gave approval for the procedure.

Neointestine Manufacture and Transplantation

Microporous polymer cylinders fashioned from non-woven polyglycolic acid sheets (Smith and Nephew, Heslington, York, UK) were treated with 5% poly-L-lactic acid and 0.1% collagen solution (Vitrogen 100, Collagen Corp, Palo Alto, Calif.) (Mooney et al. (1996) Biomaterials 17(2): 115). Intestinal organoid units were produced as described in Example 1. Polymer scaffolds, each seeded with 100,000 organoid units, were paratopically transplanted into 23 adult recipient's omental tissue under pentobarbital anesthesia. Animals were sacrificed by anesthetic overdose 1-8 weeks after implantation.

Statistics

All comparisons were analyzed for significance (p<0.05) using ANOVA.

Results

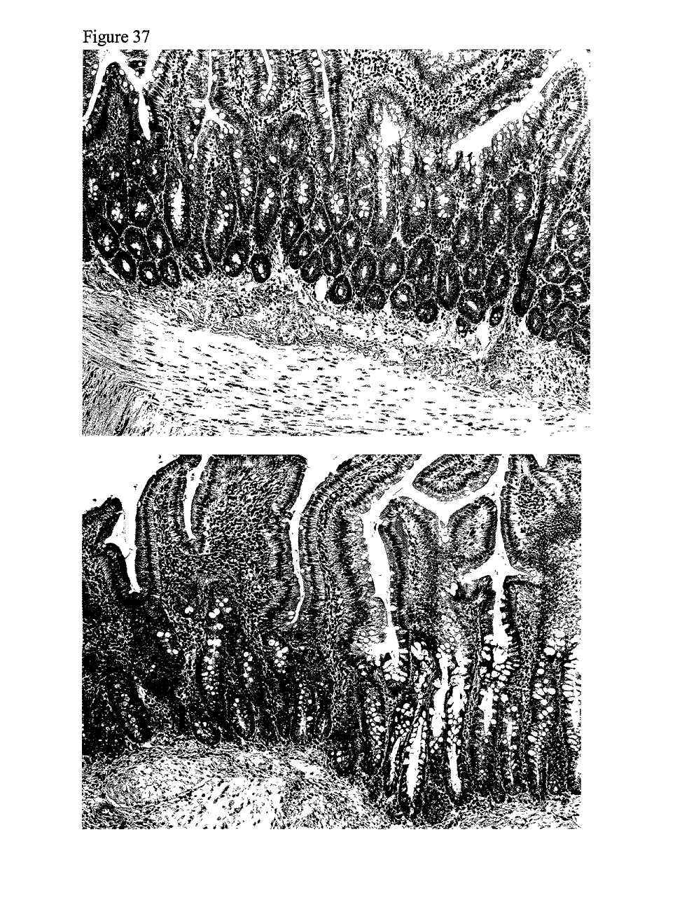

Staining and histology 5 μm sections cut from the paraffin blocks were stained with hematoxylin and eosin (H&E). Sections of neointestine, juvenile rat bowel and adult rat bowel were compared using 40× light microscopy and computer-assisted morphometric analysis. The presence or absence of villi, crypt width and the thickness of the layers were recorded in five randomly chosen fields from each slide. These layers were (1) Inner: mucosa and submucosa and (2) Outer: muscle and connective tissue (FIG. 37).

Immunohistochemistry was performed using 1:200 dilution of mouse anti-CD34 (Human/Rat) antibody (Research Diagnostics, Inc., Flanders, N.J.). The number of capillaries (defined by Rhodin, J. A. (1968) J. Ultrastruct. Res. 25(5):452) stained in each layer was counted in five random 100×fields. The vessel density is presented as the number of capillaries serving 1000 counterstained nuclei to allow meaningful comparison of native bowel with neointestine. Anti-CD34 immunohistochemistry revealed branching capillaries.

Neointestinal Growth and Development

Nineteen implants (83%) successfully developed into neointestinal cysts. The four failures were manifest as a visible scaffold under a thin layer of omentum.

At sacrifice the neointestine was dissected from recipient tissues. The complete neointestine was weighed and two radii (a and b) were measured. Volume was calculated using the formula for an oblate spheroid: volume=⁴/₃πa²b. Tissue for use in comparisons was harvested from the mid-ileum of 10 rats of 2-8 weeks of age and from five mature rats of 200-300 g body weight. Although the cysts progressively increased in mean volume to 12.6 cm² at eight weeks and the mass increased from 1.3 g to 9.7 g, both values returned to normal over time.

Vessel Density

The capillary density in the mucosal layer was similar and not statistically different for each group (juvenile, adult and neointestine). For the muscularis, the capillary density was five-times greater in adult than juvenile intestine (Table 5).

TABLE 5

| | Juvenile bowel | Mature bowel | Neointestine |
|---|---|---|---|
| | Mean ± SEM | Mean ± SEM | Mean ± SEM |
| Capillary density in | 14.49 ± 2.77 | 75.75 ± 3.07 | 82.95 ± 4.81 |
| outer tissue layer | | | |
| (capillaries per | | *p < 0.001 | *p < 0.001 |
| 1000 cells) | | | |
| Capillary density | 74.54 ± 4.80 | 82.50 ± 7.30 | 89.54 ± 5.68 |
| in inner layer | | | |
| (capillaries per | | Not Statisti- | Not Statistically |
| 1000 cells) | | cally Signifi- | Significant |
| | | cant | |

The outer layers of the neointestine had a capillary density similar to adult, rather than juvenile bowel (p<0.001). The capillary density in the neointestine did not change significantly in either layer over the duration of the study.

Growth Factor Assays

Tissues were minced and homogenized in 5 mL extraction buffer (Ishii et al. (2001) Arch. Oral Biol. 46(1):77) which consisted of phosphate-buffered saline, 0.05% Triton-X100 and 1 mM protease inhibitor 4-2-aminoethylbenzenesulfonylfluoride (Sigma, St Louis, Mo.). The specimens were processed by sonication and centrifuged at 1500× g for 10 minutes at 4° C. The total protein concentration of the supernatant was assayed using the Bradford assay (Biorad Laboratories, Hercules, Calif.). Quantikine® ELISA assay kits for VEGF and bFGF were purchased from R&D Systems (Minneapolis, Minn.) and were used according to the manufacturer's instructions. The supernatant concentration of VEGF and bFGF is presented as pg per mg total protein. The concentrations of VEGF and bFGF measured in the tissue homogenates are shown in Table 6.

TABLE 6

| | Juvenile bowel | Mature bowel | Neointestine |
|---|---|---|---|
| | Mean ± SEM | Mean ± SEM | Mean ± SEM |
| VEGF | 147.6 ± 23.93 | 49.5 ± 3.11 | 42.3 ± 3.34 |
| pg/mg total protein | | *p < 0.001 | *p < 0.001 |
| bFGF | 315.0 ± 65.48 | 167.3 ± 25.12 | 162.3 ± 15.09 |
| pg/mg total protein | | *p < 0.05 | *p < 0.05 |

Juvenile bowel had statistically significantly higher levels of VEGF (p<0.001) and bFGF (p<0.05) than adult bowel and neointestine. The VEGF concentration in the engineered-intestine was 3.4-fold lower than in the immature bowel (p<0.001). bFGF concentration was 1.9-fold lower than expected (p<0.05) in the engineered intestine. The engineered intestine tissues contained VEGF and bFGF at the same concentrations as adult intestine. No difference in concentration of either VEGF or bFGF was observed as the neointestine grew in size.

The neointestine increased in size and grew to resemble the native small bowel. The capillary density remained constant as the tissue expanded, demonstrating a controlled increase in the absolute number of capillaries to meet nutritional demands of the neointestine. Furthermore, the microporous scaffold facilitated diffusion of gases and metabolites. Taken together, these observations indicate that angiogenesis occurred.

### Example 9

### Tissue-Engineered Pancreas

A tissue-engineered pancreas was created using biodegradable polymer constructs to transplant multicellular organoid units derived from Lewis rats into the omentum of syngenic hosts. The tissue-engineered pancreas constructs were harvested at four weeks for immunohistochemistry. All rats generated tissue-engineered pancreas.

Organoid Unit Preparation

Organoid units were harvested from pancreatectomy specimens from three day old Lewis rats, purified by differential centrifugation and enzymatically digested as described in Example 1.

Implantation of Tissue-Engineered Pancreas into Rat Omentum

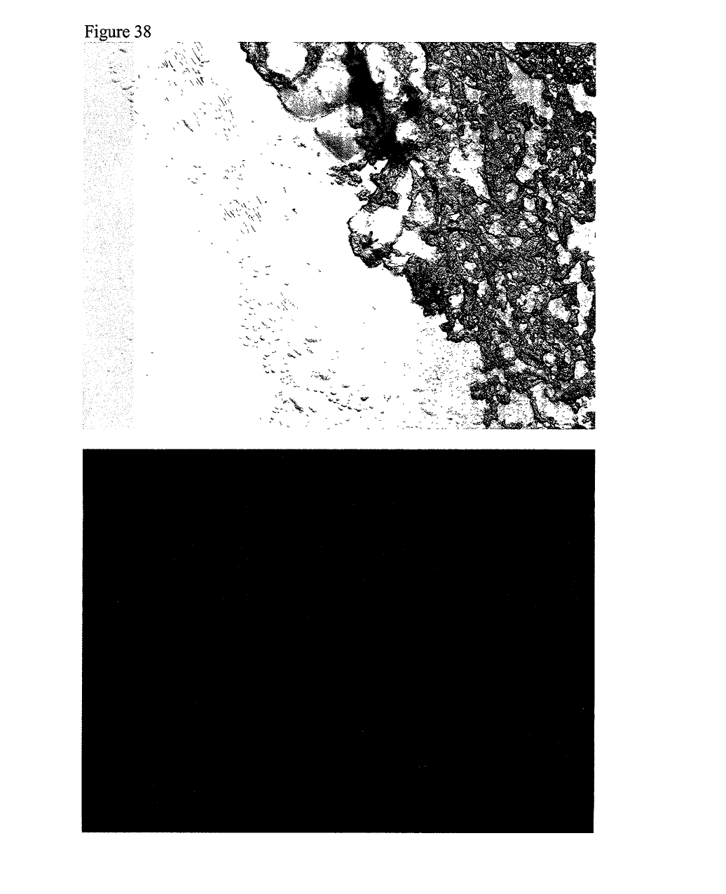

Pancreas organoid units were seeded on collagen-coated 1 cm long 0.5 mm woven polyglycolic acid tubes with a diameter of 0.5 cm. Tissue-engineered pancreas constructs were implanted in the omentum of six male Lewis rats. Implantation was achieved as described in Example 1. Care was taken to implant the tissue-engineered pancreas far from the native pancreas and to impose the liver between the tissue-engineered pancreas and native pancreas to physically separate them. The implantation was also performed with pancreas organoid units that had been labeled with GFP for one hour at 37° C. to distinguish and verify engineered tissue from host tissue (FIG. 38). Confirmation of GFP production was obtained by simultaneous in vitro culture of the organoid units. Tissue-engineered pancreas constructs were harvested after four weeks.

Results

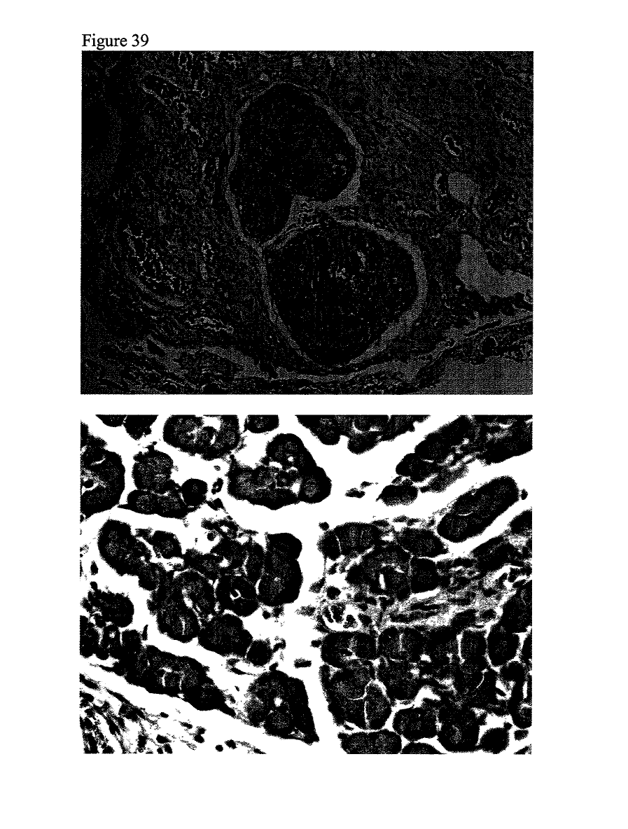

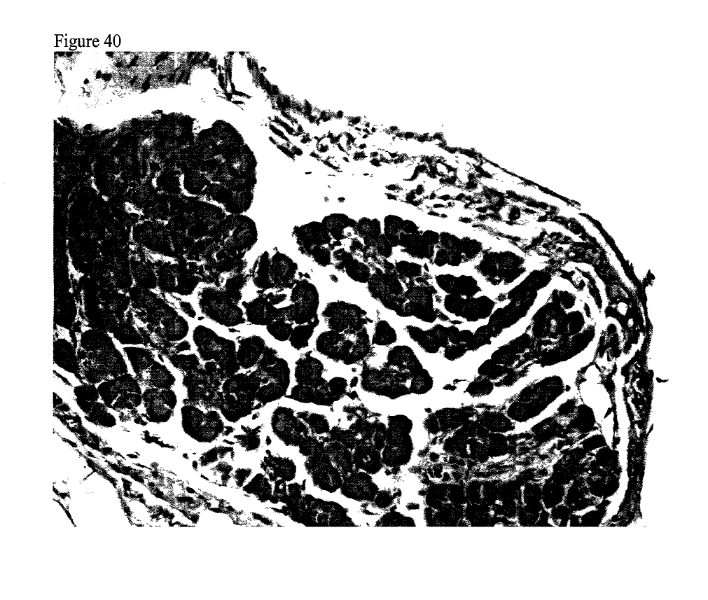

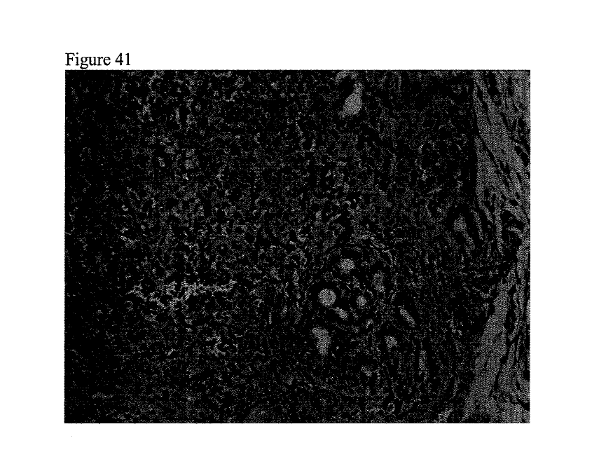

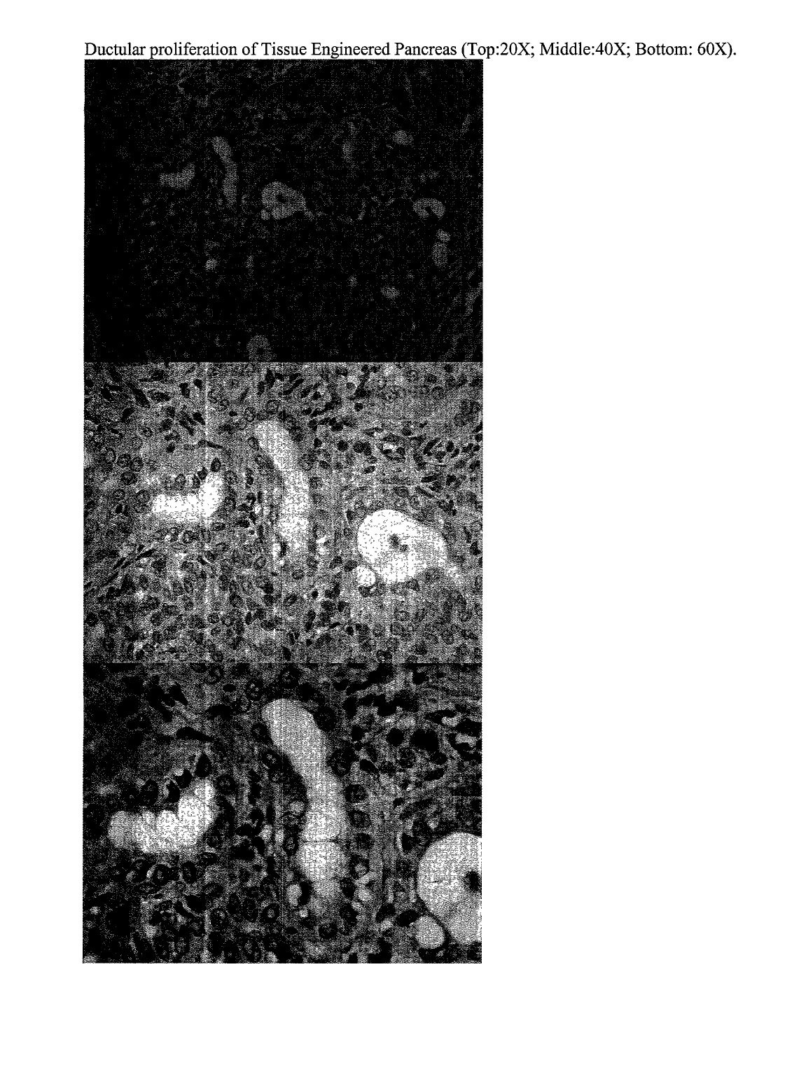

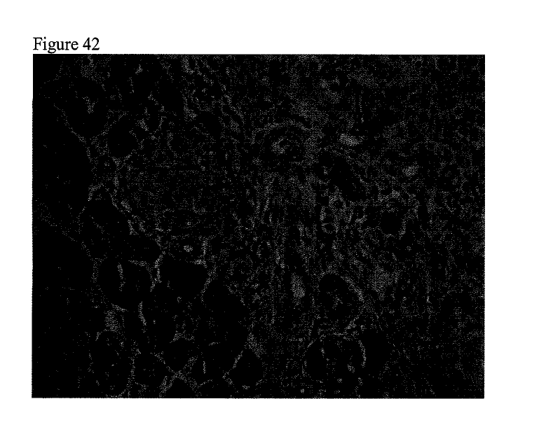

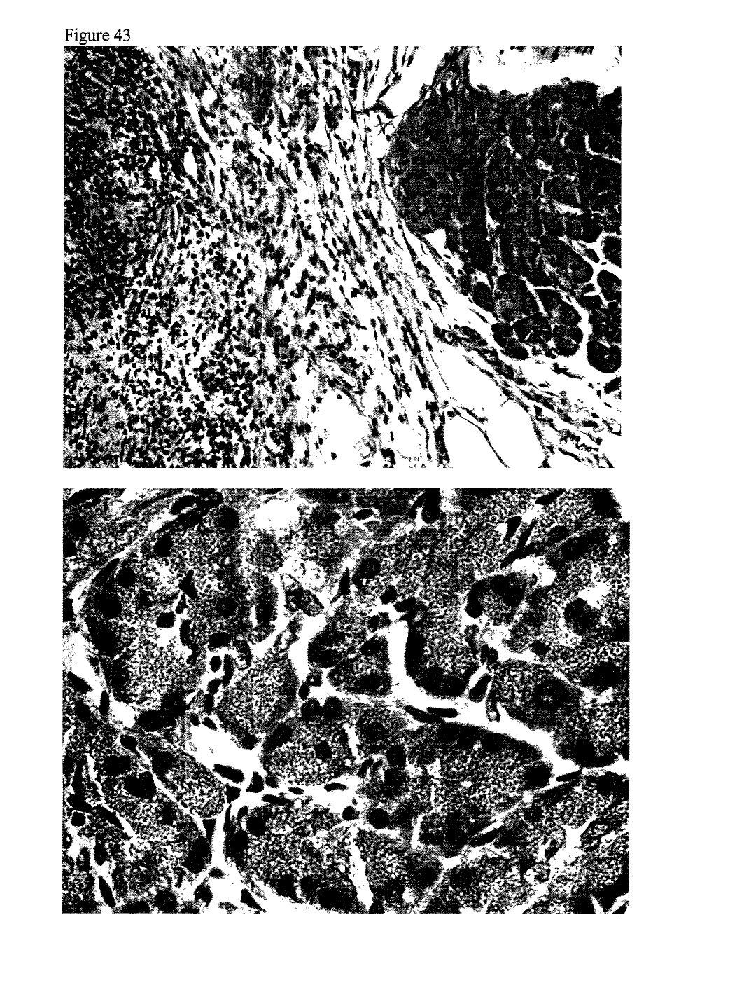

All rats generated a light beige soft tissue that encompassed the polymer within the omentum, indicative of tissue-engineered pancreas (FIG. 39). A loose tissue-engineered pancreas organization was seen with surrounding fibrosis, indicating that this was indeed the area of implantation and not the native pancreas (FIG. 40). This surrounding inflammation can be seen with all polymer implants. Disordered collections of ductal structures, produced with fidelity to make a functional pancreas (FIG. 41), and islet cells (FIG. 42) as well as acinar structures (FIG. 43) were identified by histology.

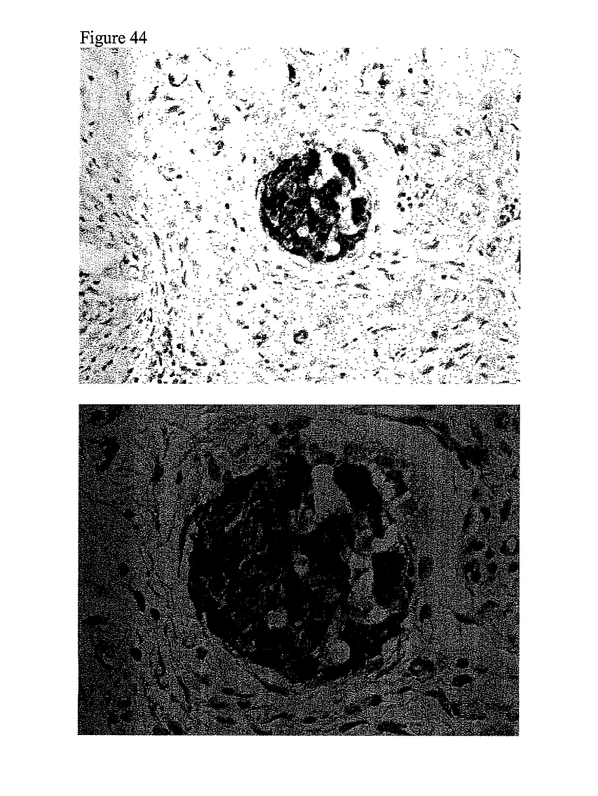

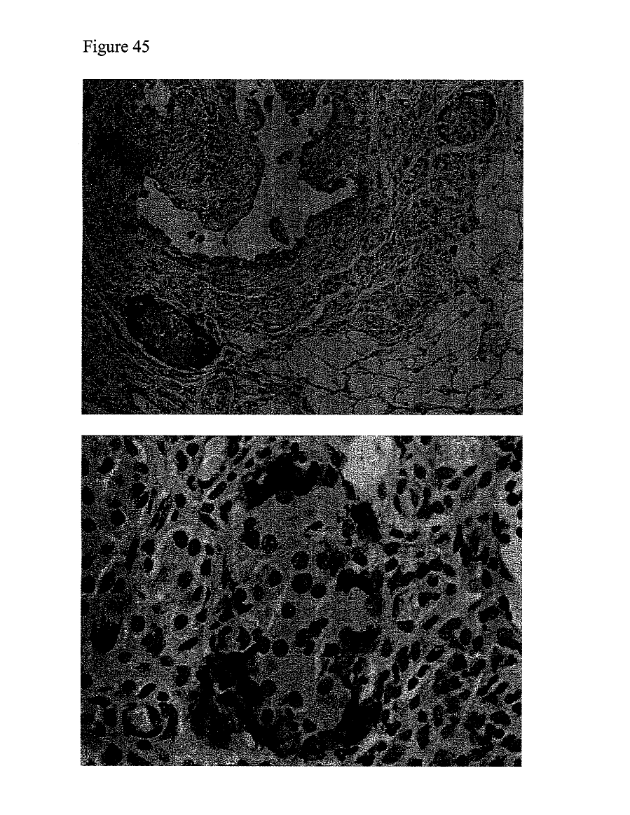

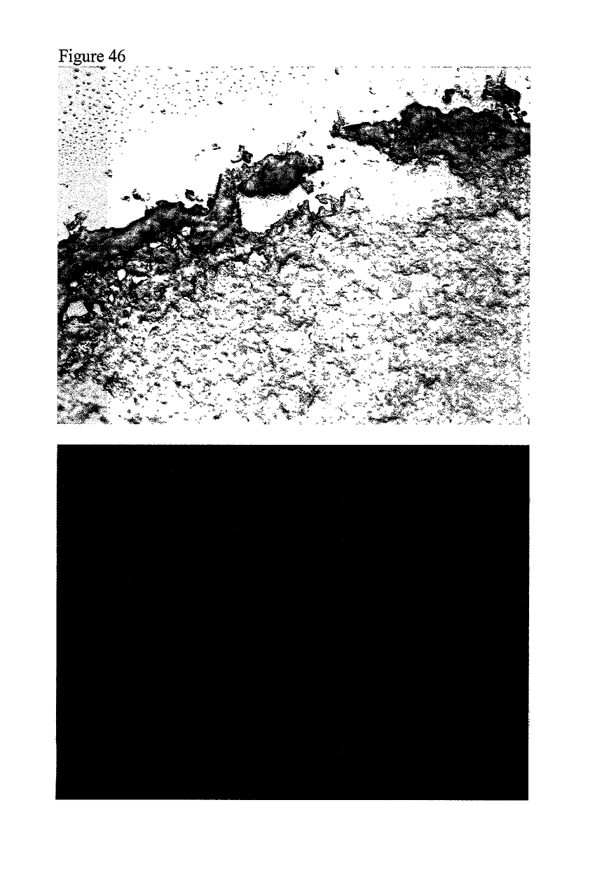

Insulin positive staining cells (FIG. 44) and glucagon positive staining cells (FIG. 45) were identified on immunohistochemical analysis of paraffin sections.

The organoid unit transfer technique can apply to the generation of tissue-engineered pancreas. These results indicate that this technique can also apply to the generation of other tissue-engineered endocrine organs, including but not limited to the thyroid, parathyroid, pituitary, hypothalamus, gonads, salivary glands and adrenal glands.

### Example 10

### Tissue-Engineered Stomach Forms from Autologous Organoid Unit Transplantation

Tissue-engineered stomach was generated by the transplantation of autologous organoid units on a polymer scaffold. The recapitulation of native stomach architecture that persisted in anastomosis points to a replacement technique that complements total gastrectomy.

Animals

Nonfasted 7-day-old neonatal Lewis rats or adult rats (Charles River Laboratories, Wilmington, Mass.) were used as stomach donors for isolation of the stomach organoid units. Adult Lewis rats (200-250 g, Charles River Laboratories) were used as recipients. Animals were housed in the Animal Research Facility of the Massachusetts General Hospital, Boston, Mass., in accordance with the National Institute of Health guidelines for care of laboratory animals. Animals were maintained in a temperature-regulated environment on a 12-hr light-dark cycle, housed in cages with soft bedding and cover, and given access to rat chow and tap water ad libitum. The health of all recipients was checked daily for the duration of this study following implantation.

Polymer Fabrication

Microporous biodegradable polymer tubes (length=10 mm, outer diameter=5 mm, inner diameter=2 mm) were made from a nonwoven mesh of PGA fibers (fiber diameter: 15 μm; mesh thickness: 2 mm; bulk density: 60 mg/cm³; porosity: >95%) (Smith and Nephew, Heslington, York, UK), coated with 5% poly (L-lactic acid) (Sigma-Aldrich, St Louis, Mo.) in chloroform. The poly (L-lactic acid) penetrates and coats the PGA mesh and also spans part of the interfibrillar space. Prior to seeding, the polymer tubes were coated with type I collagen (Vitrogen 100, Cohesion, Palo Alto Calif.).

Organoid Unit Preparation

The animals were anesthetized with ketamine/xylazine. Whole stomachs were harvested, stripped of the greater and lesser omentum, and placed in Hanks Balanced Salt Solution (HBSS) (Cellgro, Herndon, Va.) on ice. The stomachs were opened, and the contents were removed. Initially, the whole stomach was refined into organoid units. Later, the area around the esophago-gastric junction, consisting of a stratified squamous epithelium, was removed, so as to only use the area consisting of columnar epithelium.

Isolation and Seeding of Stomach Epithelium Organoid Units

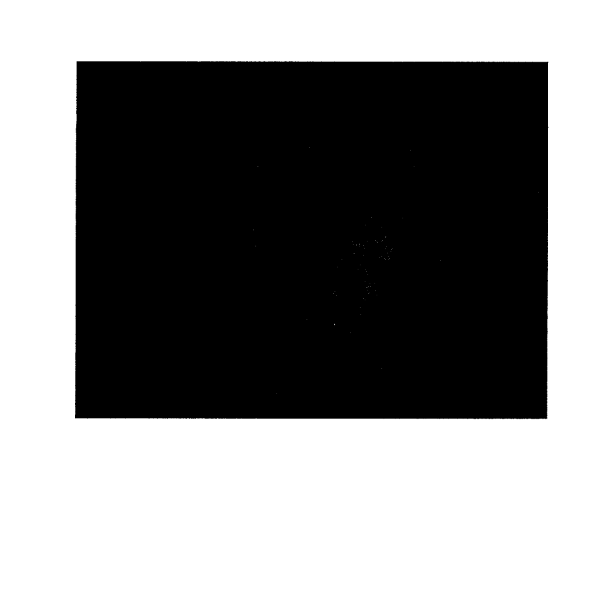

The stomach was dissected into 2 mm pieces. Tissue fragments were digested enzymatically with dispase I (0.1 mg/ml, neural protease type I, Boehringer Ingelheim, GmbH, Germany) and Collagenase XI (300 U/ml, clostridium histolycium type XI, Sigma-Aldrich, St. Louis, Mo.) at 37 degrees on an orbital shaking platform at 80 cycles/min for 25 minutes. The digestion was immediately stopped with three 4° C. washes of a solution of high glucose Dulbecco's Modified Eagle Medium (DMEM), 4% iFBS, and 4% sorbitol. The organoid units were centrifuged between washes at 150 g for five minutes, and the supernatant removed. Organoid units were reconstituted in high glucose DMEM with 10% iFBS, counted by hemocytometer, and loaded 100,000 units per polymer at 4° C., maintained at that temperature until implantation, which occurred in under 1.5 hours. In an additional preparation, the same procedure was followed, but after the organoid units were isolated, they were incubated for 2 hours with the GFP virus (FIG. 46). 200,000 OU were maintained in a 12-well plate to measure GFP production in vitro, and the remainder of the organoid units were implanted. GFP detection two weeks after anastomosis was performed on 10 micron frozen section without fixation and with native tissue controls.

Implantation of Seeded Polymer Tube and Anastomosis

Adult Lewis rats (n=14) were used as recipients for implantation of stomach organoid unit/polymer constructs. Following ketamine/xylazine anesthesia, the recipients underwent an upper midline incision and the omentum was exposed. The seeded polymer tubes were wrapped completely into the omentum, secured with suture, placed back into the abdominal cavity, and the abdominal cavities were closed in two layers. Three weeks after the initial implantation, the polymer tubes seeded with organoid units formed Tissue-Engineered Stomach (TES). Nine rats underwent a second operation for anastomosis. TES was anastomosed to the native jejunum at the 5 cm distal site to the ligament of Treitz in a side-to-side fashion with suture. The body weights of all animals were measured twice a week for 8 weeks following anastomosis. The changes in body weight were compared with those of normal rats.

Results

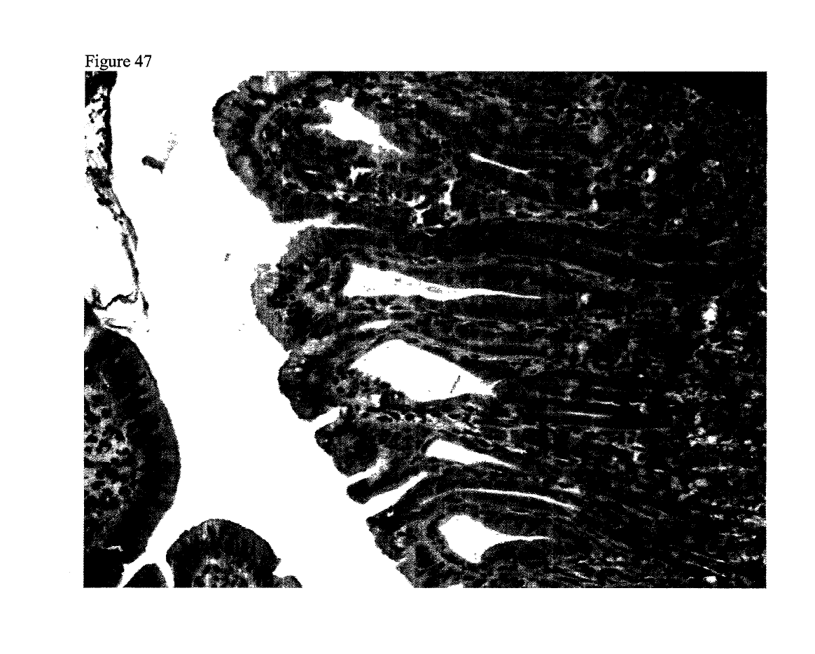

Animals transplanted with stomach organoid unit/polymer contructs were sacrificed at 2, 4, 6, and 8 weeks post-implantation and the TES constructs were harvested. Anastomosed animals were harvested 2-4 weeks after anastomosis, which occurred 4 weeks after implantation. TES formed in 98% of all implantations, including both neonatal and adult donor origin (FIG. 47). The dimensions (length and diameter) of the TES constructs were measured. Cysts averaged 3×5 cm in size. There were no statistically significant differences between the groups. The average length and diameter were larger compared to a native stomach.

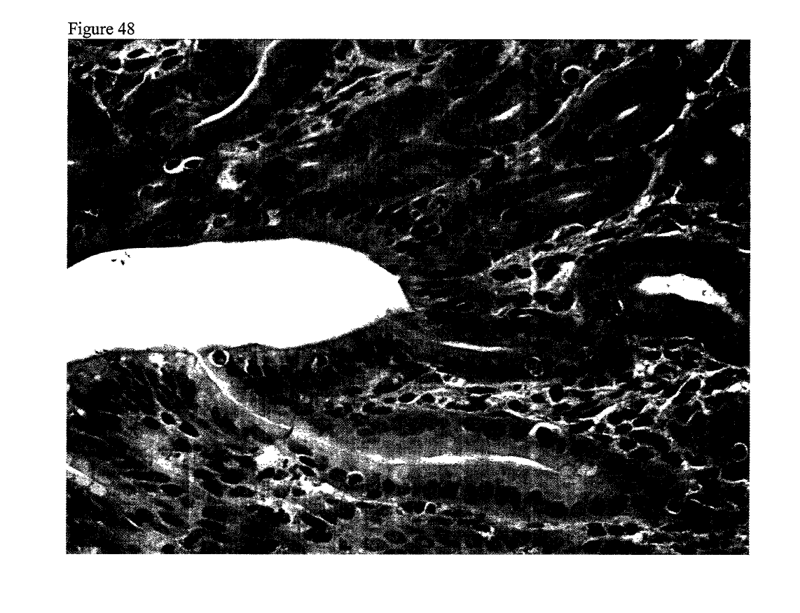

All specimens were fixed in 10% formalin solution, parafiin-embedded, and 4 μm sections were stained with hematoxylin and eosin for histological assessment. The lumen of the tissue-engineered stomach was filled with thick mucus and debris. At 2 weeks, there were areas lacking a mucosal layer and were occupied with fibrous tissue, some inflammatory cells, and degrading polymer. The H&E stained histologic sections of the TES (FIG. 48) at 4, 6, and 8 weeks showed that while some parts still lacked a mucosal layer, other parts showed the development of vascularized tissue with a neomucosa lining the lumen. The villi of these tissue-engineered stomachs were shorter than those of native stomachs. Though neomucosa was composed of columnar epithlium surrounded by a wall of vascularized tissue, extracellular matrix and smooth muscle-like cells, no sub-mucosal layer was evident.

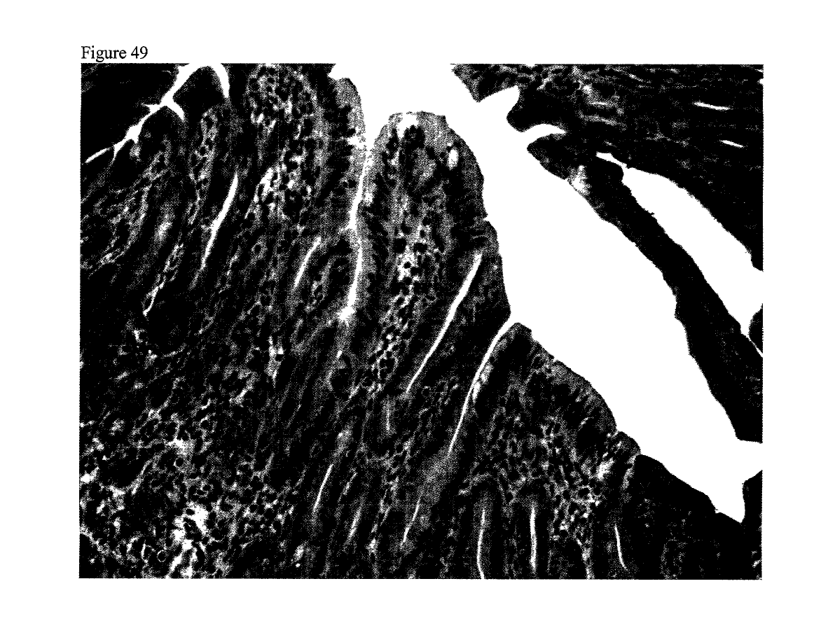

In the anastomosed animal, all animals showed weight loss immediately after surgery, but gradually returned to their normal weights by the third week after anastomosis. The anastomosed cysts were slightly smaller compared to those before anastomosis, but retained their lumen. Histology of the tissue-engineered stomach was also maintained in the anastomosed animals (FIG. 49). The surface had a slight gap at the anastomosis site, but was well covered with the epithelium that originated from organoid units. Anastomosis between the TES and the native small intestine stimulated the growth of epithelium.

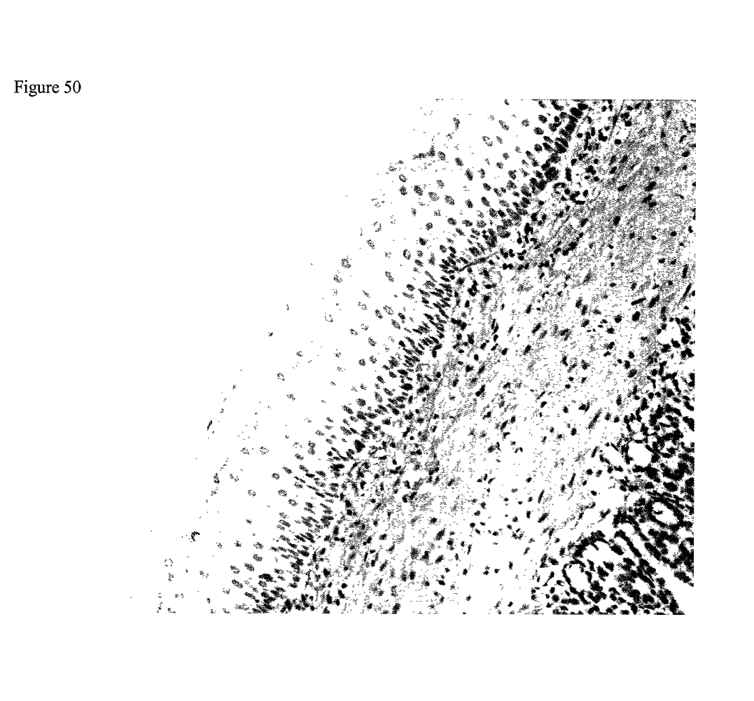

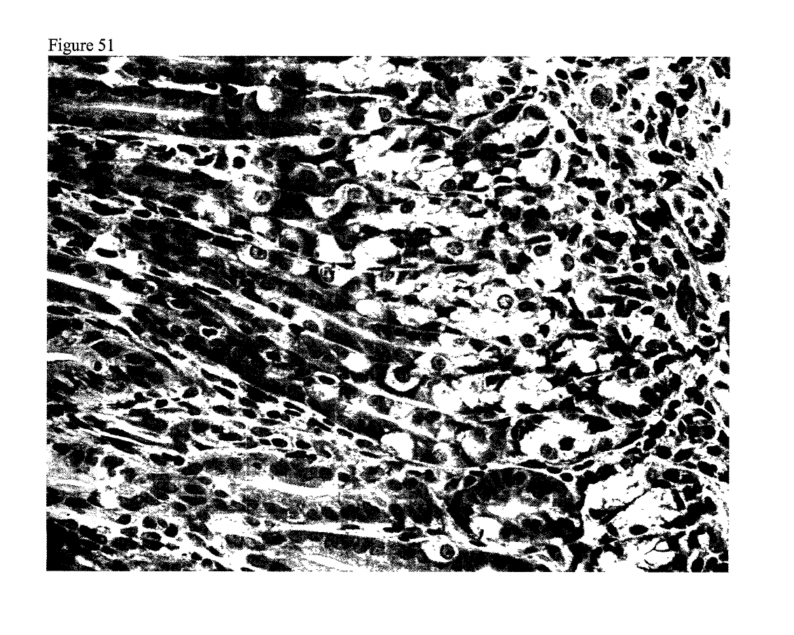

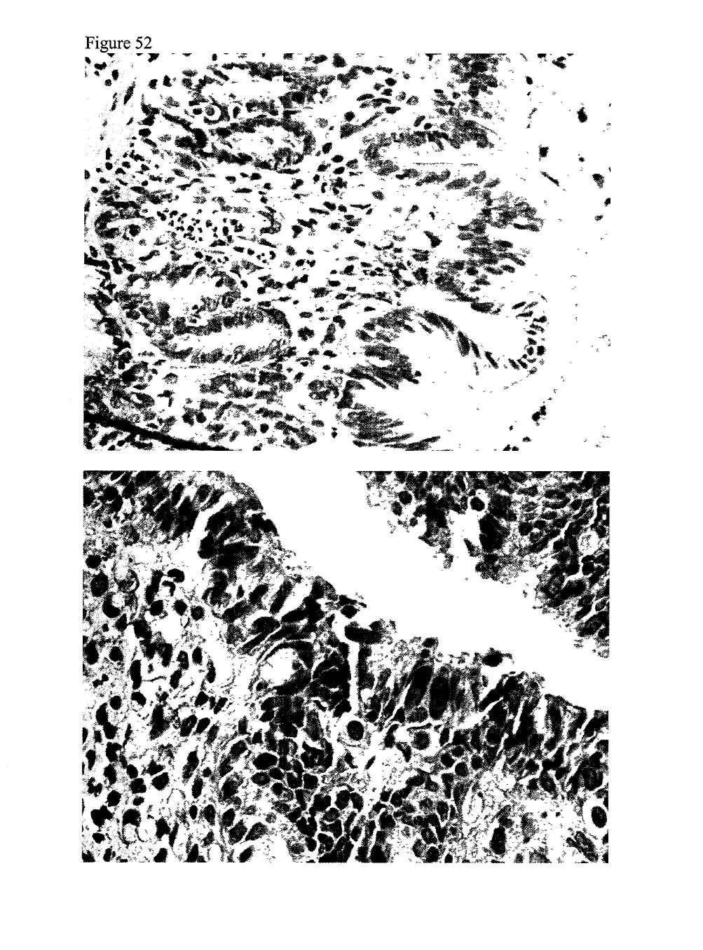

Immunohistochemical staining was performed to identify cell surface markers for gastric mucin (Clone: 45M1, Sigma, St. Louis) and α-actin smooth muscle (Clone: 1A4, DAKO, Carpinteria, Calif.) to identify the localization of the columnar epithelium and the smooth muscle layer. A labeled Streptavidin-Biotin system (DAKO, Carpinteria, Calif.) was used for immunohistochemical staining with appropriate controls. Immunohistochemical staining for α-actin smooth muscle was positive in the stroma adjacent to the neomucosa, confirming the presence of smooth muscle layers (FIG. 50). Immunohistochemical staining for gastric mucin was positive on the surface of the gastric epithelium, indicating a well developed gastric epithelium (FIG. 51). In addition, immunohistochemical staining for gastrin was positive, indicating intact APUD (Amino Precursor Uptake and Decarboxylase) cells (FIG. 52).

Scanning electron microscopy was performed and revealed a villous pattern on the surface of the gastric epithelium. However, the surface structure was rougher than that of a native stomach with shorter villi.

Tissue-engineered stomach formed from full dissection of the stomach without exclusion of the area around the esophago-gastric junction, consisting of a stratified squamous epithelium, which consisted of cysts with measurements that were not statistically significantly different from those that excluded the esophago-gastric junction but were composed of intermixed segments of stratified squamous epithelium and gastric glands.

Tissue-engineered stomach formed from donor tissue carrying the GFP marker maintained GFP signal on frozen sections after four weeks of growth and 2 weeks of anastomosis to small intestine. Native stomach was not autofluorescent. These results demonstrate that the tissue-engineered stomach was generated solely from donor organoid units and not from the host tissue.

Tissue-engineered stomach formed from transplanted stomach organoid units is a complex tissue resembling native stomach.

### Example 11

### Tissue-Engineered Cardiac Muscle

Cardiac muscle wall defects occur after myocardial infarction, penetrating trauma, and in the case of congenital malformations or conjoined twins. Although, over time, skeletal muscle applied in a wrap on the heart takes on some aspects of cardiac muscle, no suitable muscle substitution has yet been generated. This is a case in which surgical repair by proxy is not possible.

The object of this procedure is the generation of usable cardiac muscle in a pedicle in the omentum from transplantation of syngeneic or autologous musle via cardiac organoid units, which could be applied over the area of the defect. Myocardial infarction most often leads to destruction in the area supplied by the Left Anterior Descending branch, and that is anterior on the heart, the most accessible area to reach via an omental pedicle, so no change in implantation or harvesting procedure over what has been described in the previous Examples is necessary. The approach can be either intra- or extra-thoracic.

Organoid Unit Preparation

Organoid units were harvested from the full thickness hearts of three day old Lewis rats, purified by differential centrifugation and enzymatically digested as described in Example 1.

Implantation of Tissue-Engineered Cardiac Muscle into Rat Omentum

Cardiac organoid units were seeded on collagen-coated 1 cm long 0.5 mm woven polyglycolic acid tubes with a diameter of 0.5 cm. Tissue-engineered cardiac constructs were implanted in the omentum of four male Lewis rats. Implantation was achieved as described in Example 1. Tissue-engineered cardiac muscle constructs were harvested after four weeks.

Results

Numerous cells comprising the myocardium were viewed in a cross section. Centrally placed nuclei—a key feature of cardiac muscle—were observed.

### Example 12

### Tissue-Engineered Kidney

Organoid Unit Preparation

Organoid units were harvested from the kidney of Lewis rats, purified by differential centrifugation and enzymatically digested as described in Example 1.

Implantation of Tissue-Engineered Cardiac Muscle into Rat Omentum

Kidney organoid units were seeded on collagen-coated 1 cm long 0.5 mm woven polyglycolic acid tubes with a diameter of 0.5 cm. Tissue-engineered kidney constructs were implanted in the omentum of Lewis rats. Implantation was achieved as described in Example 1. Tissue-engineered kidney constructs were harvested after four weeks.

Results

Collecting tubules and loop of Henle were observed.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. A method for producing a tissue-engineered organ or portion thereof comprising the steps of:
(a) loading organoid units into a biocompatible polymer scaffold at a high density; and
(b) implanting the polymer scaffold into a subject,
wherein the organoid units are derived from a tissue selected from the group consisting of spleen, lung, liver, kidney, pancreas, endocrine, heart, esophagus, colon, stomach, gall bladder and uterus, and wherein the organ or organ portion is of the same tissue type as the organoid unit from which it is produced.

2. The method of claim 1, wherein the organoid units are derived from spleen and the organ is spleen.

3. The method of claim 1, wherein the organoid units are derived from lung and the organ is lung.

4. The method of claim 1, wherein the organoid units are derived from liver and the organ is liver.

5. The method of claim 1, wherein the organoid units are derived from kidney and the organ is kidney.

6. The method of claim 1, wherein the organoid units are derived from pancreas and the organ is pancreas.

7. The method of claim 1, wherein the organoid units are derived from endocrine tissue and the organ is an endocrine gland.

8. The method of claim 7, wherein the endocrine gland is selected from the group consisting of thyroid, parathyroid, pituitary, hypothalamus, gonads, salivary glands and adrenal glands.

9. The method of claim 1, wherein the organoid units are derived from heart and the organ portion is cardiac muscle.

10. The method of claim 1, wherein the organoid units are derived from esophagus and the organ is esophagus.

11. The method of claim 1, wherein the organoid units are derived from colon and the organ is colon.

12. The method of claim 1, wherein the organoid units are derived from stomach and the organ is stomach.

13. The method of claim 1, wherein the organoid units are derived from gall bladder and the organ is gall bladder.

14. The method of claim 1, wherein the organoid units are derived from uterus and the organ is uterus.

15. The method of any of claims 1-14, wherein the loading of the organoid units is at a density of at least 100,000 units per 1.5 cm² polymer scaffold.

16. The method of claim 1, wherein the implanting is into the omentum.

17. The method of claim 1, wherein the polymer scaffold is tubular.

18. The method of claim 1, wherein the polymer scaffold has at least 95% porosity.

19. The method of claim 1, wherein the polymer scaffold has a coating.

20. The method of claim 19, wherein the coating is a material selected from the group consisting of agar, agarose, basement membrane material, collagen types I, II, II, IV and V, fibronectin, gelatin, glycosaminoglycans, gum arabic, laminin, poly-L-lactic acid and mixtures thereof.

21. The method of claim 1, wherein the polymer scaffold is biodegradable.

22. The method of claim 21, wherein the polymer scaffold comprises a material selected from the group consisting of polylactide, poly-L-lactide, poly-D-lactide, polyglycolide, polyglycolic acid, polylactide-co-glycolide, polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, oligo(ε-caprolactone)diol/oligo(p-dioxyanone)diol and aliphatic polyesters.

23. The method of claim 22, wherein the material is polyglycolic acid.

24. The method of claim 1, wherein the polymer scaffold is non-biodegradable.

25. The method of claim 24, wherein the polymer scaffold comprises a material selected from the group consisting of polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, teflon RTM, nylon, silicon, poly(styrene-block-butadiene), polynorbomene, hydrogels and metallic alloys.

26. The method of claim 1, wherein the organoid units are prepared by the steps of:
(a) digesting pieces of tissue with an enzyme or enzyme mixture; and
(b) purifying the tissue to obtain the organoid units.

27. The method of claim 26, wherein the tissue is harvested from previously engineered tissue.

28. The method of claim 26, wherein the tissue is harvested from a donor.

29. The method of claim 28, wherein the donor is neonatal, juvenile or adult.

30. The method of claim 28, wherein the donor is the subject.

31. The method of claim 26, wherein the enzyme mixture is dispase/collagenase.

32. The method of claim 31, wherein digesting is performed at about 37° C. for about 20 minutes, and wherein the organoid units are derived from a tissue selected from the group consisting of spleen, lung, liver, kidney, pancreas, endocrine and heart.

33. The method of claim 31, wherein digesting is performed at about 37° C. for about 35 minutes, and wherein the organoid units are derived from a tissue selected from the group consisting of esophagus, colon, stomach, gall bladder and uterus.

34. The method of claim 1, wherein the organoid units express GFP.

35. A method for producing tissue-engineered duodenum, jejunum or ileum comprising the steps of:
(a) preparing organoid units by
(i) obtaining pieces of duodenal tissue to engineer duodenum, pieces of jejunal tissue to engineer jejunum or pieces of ileal tissue to engineer ileum; and
(ii) digesting the tissue obtained in (i) with an enzyme; and
(iii) purifying the tissue to obtain organoid units; and
(b) loading the organoid units into a biocompatible polymer scaffold at high density; and
(c) implanting the polymer scaffold into a subject.

36. A tissue-engineered organ produced by the method of claim 1.

37. A tissue-engineered spleen produced by the method of claim 2.

38. A tissue-engineered lung produced by the method of claim 3.

39. A tissue-engineered liver produced by the method of claim 4.

40. A tissue-engineered kidney produced by the method of claim 5.

41. A tissue-engineered pancreas produced by the method of claim 6.

42. A tissue-engineered endocrine gland produced by the method of claim 7.

43. Tissue-engineered cardiac muscle produced by the method of claim 9.

44. A tissue-engineered esophagus produced by the method of claim 10.

45. A tissue-engineered colon produced by the method of claim 11.

46. A tissue-engineered stomach produced by the method of claim 12.

47. A tissue-engineered gall bladder produced by the method of claim 13.

48. A tissue-engineered uterus produced by the method claim 14.

49. A tissue-engineered duodenum produced by the method of claim 35.

50. A tissue-engineered jejunum produced by the method of claim 35.

51. A tissue-engineered ileum produced by the method of claim 35.

52. A tissue-engineered organ or portion thereof comprising compact tissue grown in a biocompatible polymer scaffold, wherein the tissue is derived from spleen, lung, liver, kidney, pancreas, endocrine, heart, esophagus, colon, stomach, gall bladder or uterus.

53. The organ of claim 52, wherein the tissue is derived from spleen and the organ is spleen.

54. The organ of claim 52, wherein the tissue is derived from lung and the organ is lung.

55. The organ of claim 52, wherein the tissue is derived from liver and the organ is liver.

56. The organ of claim 52, wherein the tissue is derived from kidney and the organ is kidney.

57. The organ of claim 52, wherein the tissue is derived from pancreas and the organ is pancreas.

58. The organ of claim 52, wherein the organoid units are derived from endocrine tissue and the organ is an endocrine gland.

59. The organ of claim 58, wherein the endocrine gland is selected from the group consisting of thyroid, parathyroid, pituitary, hypothalamus, gonads, salivary glands and adrenal glands.

60. The organ part of claim 52, wherein the tissue is derived from heart and the organ portion is cardiac muscle.

61. The organ of claim 52, wherein the tissue is derived from esophagus and the organ is esophagus.

62. The organ of claim 52, wherein the tissue is derived from colon and the organ is colon.

63. The organ of claim 52, wherein the tissue is derived from stomach and the organ is stomach.

64. The organ of claim 52, wherein the tissue is derived from gall bladder and the organ is gall bladder.

65. The organ of claim 52, wherein the tissue is derived from uterus and the organ is uterus.

66. The organ of claim 52, wherein the polymer scaffold is tubular.

67. The organ of claim 52, wherein the polymer scaffold has at least 95% porosity.

68. The organ of claim 52, wherein the polymer scaffold has a coating.

69. The organ of claim 68, wherein the coating is a material selected from the group consisting of agar, agarose, basement membrane material, collagen types I, II, II, IV and V, fibronectin, gelatin, glycosaminoglycans, gum arabic, laminin, poly-L-lactic acid and mixtures thereof.

70. The organ of claim 52, wherein the polymer scaffold is biodegradable.

71. The organ of claim 70, wherein the polymer scaffold comprises a material selected from the group consisting of polylactide, poly-L-lactide, poly-D-lactide, polyglycolide, polyglycolic acid, polylactide-co-glycolide, polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, oligo(ε-caprolactone)diol/oligo(p-dioxyanone)diol and aliphatic polyesters.

72. The organ of claim 71, wherein the material is polyglycolic acid.

73. The organ of claim 52, wherein the polymer scaffold is non-biodegradable.

74. The organ of claim 73, wherein the polymer scaffold comprises a material selected from the group consisting of polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, teflon RTM, nylon, silicon, poly(styrene-block-butadiene), polynorbomene, hydrogels and metallic alloys.

75. The organ of claim 52, wherein the tissue is harvested from previously engineered tissue.

76. The organ of claim 52, wherein the tissue is harvested from a donor.

77. The organ of claim 76, wherein the donor is neonatal, juvenile or adult.

78. The organ of claim 52, wherein the organoid units express GFP.

79. The method of claim 1, wherein the subject is a human.

80. The method of claim 35, wherein the subject is a human.

81. The method of claim 26, wherein the tissue is derived from spleen and the organ is spleen.

82. The method of claim 26, wherein the tissue is derived from lung and the organoid unit comprises lung tissue.

83. The method of claim 26, wherein the tissue is derived from liver and the organoid unit comprises liver tissue.

84. The method of claim 26, wherein the tissue is derived from kidney and the organoid unit comprises kidney tissue.

85. The method of claim 26, wherein the tissue is derived from pancreas and the organoid unit comprises pancreatic tissue.

86. The method of claim 26, wherein the tissue is derived from an endocrine gland and the organoid unit comprises endocrine tissue.

87. The method of claim 86, wherein the endocrine gland is selected from the group consisting of thyroid, parathyroid, pituitary, hypothalamus, gonads, salivary glands and adrenal glands.

88. The method of claim 26, wherein the tissue is derived from heart and the organoid unit comprises cardiac tissue.

89. The method of claim 26, wherein the tissue is derived from esophagus and the organoid unit comprises esophageal tissue.

90. The method of claim 26, wherein the tissue is derived from colon and the organoid unit comprises is colonic tissue.

91. The method of claim 26, wherein the tissue is derived from stomach and the organoid unit comprises stomach tissue.

92. The method of claim 26, wherein the tissue is derived from gall bladder and the organoid unit comprises gall bladder tissue.

93. An organoid unit produced according to the method of claim 81.

94. An organoid unit produced according to the method of claim 82.

95. An organoid unit produced according to the method of claim 83.

96. An organoid unit produced according to the method of claim 84.

97. An organoid unit produced according to the method of claim 85.

98. An organoid unit produced according to the method of claim 86.

99. An organoid unit produced according to the method of claim 87.

100. An organoid unit produced according to the method of claim 88.

101. An organoid unit produced according to the method of claim 89.

102. An organoid unit produced according to the method of claim 90.

103. An organoid unit produced according to the method of claim 91.

104. An organoid unit produced according to the method of claim 92.
